SEQUENCE LISTING (24 sequences)

SEQ ID NO: 1 - DNA, 1297 bp, L. vannamei
aaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggttgtttgccggatcaagagctaccaactctttttccgaagtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaactcgcctctccccgcgcgttggccgattcattaatgcagctggcacgacaggtttcccaactggaaagcgggcagtgagcgcaacccaattaatgtgagttagctcactcattaggcaccccaggctttacactttatgcttccggctcgtatgttgtgtggaattgtgagcggataacaatttcacacaggaaacagctatgaccatgattacgccaagctcgaaattaaccctcactaaagggaacaaaagctggagctccccaccgcggtggcggccgctctagaactagtggatcccccgggctgcaggaatttggcacgagctgccgaattcggcacgagcccgagaggaagcagcacgtacgctcgtccgcccttttagtaaaacaacaacaacaag

SEQ ID NO: 2 - DNA, 1249 bp, L. vannamei
atgtgtgacgacgaagtagccgccctggttgtagacaatggctccggcatgtgcaaggccggcttcgctggtgacgatgcaccacgagctgtgttcccctccatcgtcggccgaccccgtcatcagggtgtgatggtcggcatgggccagaaggactcgtacgtcggcgacgaggcccagagcaagcgaggtatcctcaccctgaaataccccatcgagcacggcatcgtcaccaactgggacgacatggagaagatctggcatcacactttctacaacgagctccgcgtggcccccgaggagcaccccgtcctgctgaccgaggctcccctcaaccccaaggctaaccgcgagaagatgacacagatcatgttcgagaccttcaacacccccgccatgtacgtggccatccaggccgtgctgtccctgtacgcctccggccgtaccaccggtatcgtgctcgactccggcgacggcgtgtcccacaccgtgcccatctacgagggatatgccctgccccacgccatcctgcgtctggacttggccggccgcgacctcacagactacctgatgaagatcctgacggagcgtggctacaccttcacgaccaccgccgagcgagaaatcgttcgtgacatcaaggagaaactgtgctacgtggccctggacttcgagcaggagatgaccaccgctgcttcctcctcctcgctggagaagtcctacgagctccctgacggccaggtgatcaccatcggcaacgagaggttccgctgccccgaggccctgttccagccctcattcctgggcatggaatcctgcggcatccacgagaccacctacaactccatcatgaagtgcgacgtggacatccgtaaggacctgtacgccaacaccgtgctgtccggaggcaccaccatgtaccctggcatcgccgacaggatgcagaaggaaatcactgccctcgctccctccaccatgaagatcaagatcatcgccccacccgagcgcaagtactccgtgtggatcggcggctccatcctggcctcgctctccaccttccagcagatgtggatcagcaagcaggagtacgacgagtctggaccatcaattgttcacaggaagtgcttctaattaacaaaatgtacatgatataggctacactttttacatttaattattccattaggataaggattatgttatttaaaaggaataaattcatttctacaaaaaaaaaaaaaaaaaaaaa

SEQ ID NO: 3 - protein, 376 aa, L. vannamei
MCDDEVAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVLDSGDGVSHTVPIYEGYALPHAILRLDLAGRDLTDYLMKILTERGYTFTTTAEREIVRDIKEKLCYVALDFEQEMTTAASSSSLEKSYELPDGQVITIGNERFRCPEALFQPSFLGMESCGIHETTYNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 4 - DNA, 2200 bp, L. vannamei
ggactcgatctggccatccctcttggctcgatgtcgcattcttgggtagtagcgtagggctagttcgcggtaagtctgtataaaagggtgcacctgcctctaaccaggtagtcgtgtcaaggctcaaacccgggtaagtgcaacgtgacacaaagcgtggctcaggtgcggcgaaaaggtgcaagtgtgtgttgaggtgaaaataaaagtggtgtggaggattaagaaggttgctggacgtcacttatcattgtgtgaacatttagtatggattataaatcagttgctttcatatgtatgtgtatctagaagtacgtgtggtttatgccattgtgaaagaggtttaagagaaacagaaagtaatggagatgtaattacttttagttgctgaatattgcaaacggttacttgagcacgaggacatctttatacattgtataatcattaacatccatttcagtggttaaaataataattataatagataaaggcagttataatagacattgtctattaatgagagtcaatcagatatgctaaaaggatataggctatgcgtaattatattgcttacggaaaactatttgaaagattcacacaaaagataatatctgaactatattggattatattgaatgttgaccccacatttatatatatatatatgtatatgtatatatatgtatttatatacataaatatacacatattcatgtatatatatatgtatatatacatatatatttatatatatgcatatatatatgtatatatatatatatacatatgcatattcatatatatgtatatatatacatatacatatatatgtatatatatgcataaatatatgtatatgaatatatatatatatatacatacatacatacatatatatatatatatatatatatatatatatatatatatatatatacacacacacacatgcacgcacatatacacacacacaaacacacacacatatatatgtaaatgcatacatatgtgtacacggacacatatatacacatatatacatacacacacaacagatacatatgcatatacatacatacacatatatctctctctatatacatatatatgtatatatgtgtatatacttatacataaaaacacacatttgcgcacacacatacacaatatacgacgcatacatatgcatataaacacacacaaacacacatatatccatatctgtctatcaatgtacacatacacatatatctatatctatctattaatgcacacacacagagacacagacacacacatttatgtataatatatatatatatatatatatatatatatatatatatatatatacatatatatttatgtatatacatacagtatatacacacatacacacaaacacatttatctatatatctttatctatctatctgcctatcaatcaatcaatctatctatctatctatctataatatctatctatctatctatctatctatctatctatctatatatatgtatgtatgtatgtatacacacacacacataagcacacacacacacatacacacacacacacacatatatacacacacaatctgcctatcaatcaatcagtatatatgtacacacacacatatatatgcgcagactcataattgcacatatacacacacacatatatatacagtatatatatatatatatatatatatatatatatatatatatatatatatgtatatatatatatactcatatatacatacacacacatacacgcacacacacacacacacatatacacatgtaagttcacaggtacacagtatatatacataaattttttctctgtctttatctttatgtattaacaaatgtgtatgtatatatatcgtatatgtatatatggtatatcagattcatacatatgggtatatctataaataattaatcactcgtgtttcgtagaatgtctgtaaataggcgcaagcatgcacactcacttttaaacgcttgtgtacacctgcacgtatgcacaaaaggtgtatgtaattttacataaatactacaacagcacatacatatacatgataatgtttgggtaatctcagtaatacaaaggtatctatgaacattaatctcattttcattttatctcaacagtgtcctctgacaactcacctatatcacaatc

SEQ ID NO: 5 - DNA, 1198 bp, L. vannamei
atgtgtgacgacgaagactcgtgtgcgctcgtgtgcgacaatggctccggtatggtcaaggccggattcgcaggagacgacgcccctcgcgccgtcttcccatccatcgttggtcgtgctcgtcaccagggtgtgatggtcggtatgggtcagaaggacgcctacgttggtgatgaggcccagagcaaacgtggtatcctcaccctcaagtaccccattgagcacggtatcatcaccaactgggatgacatggagaagatctggtaccacaccttctacaatgagctccgtgttgcccctgaagagtcccccacacttctcactgaggctcccctcaaccccaaggccaaccgtgagaagatgactcagatcatgttcgagtccttcagtctccctgccatgtatgtgaccatccaggctgtgctttctctgtacgcctctggtcgtaccactggtcaggtttgcgactctggtgatggtgtgacccacatggtccccgtttatgaaggtttcgctcttcctcatgctatccttcgtgttgatttagctggtcgtgatcttaccaactacctgatgaagatcatgactgagcgtggctactccttcactaccaccgctgaacgtgaaatcgtccgtgacatcaaggagaagctttgctacatcgcccttgatttcgaaggtgagatgaacgtcgctgctgcttcctcctccttggacaagtcctacgaacttcctgacggtcaggtcatcaccatcggtaacgagcgtttccgtgctcccgaggctctgttccagccttccttccttggtatggaatctgctggtgttcaggaaaccgtgcacagctctatcatgaggtgcgacattgacatcaggaaggatctgttcgccaatattgtgctctctggtggtactactatgtaccctggtattgctgaccgcatgcagaaggaaatcacaaccttggctccttccaccatcaagatcatcgccccacccgagcgcaagtactccgtgtggatcggcggctccatcctggcctcgctctccaccttccagcagatgtggatcagcaagcaggagtacgacgagtctggaccatcaattgttcacaggaagtgcttctaaatgtaggagactgaaaacttttattacagttgataataaaatccaaaagcaaaaaaaaaaaaaaaaaaaa

SEQ ID NO: 6 - protein, 375 aa, L. vannamei
MCDDEDSCALVCDNGSGMVKGGFAGDDAPRAVFPSIVGRARHQGVMVGMGQKDAYVGDEAQSKRGILTLKYPIEHGIITNWDDMEKIWYHTFYNELRVAPEESPTLLTEAPLNPKANREKMTQIMFESFSLPAMYVTIQAVLSLYASGRTTGHVCDSGDGVTHMVPVYEGIARPHAILRVDLAGRDLTNYLMKIMTERGYSFTTTAEREIVRDIKEKLCYIALDFEGEMNVAAASSSLDKSYELPDGQVITIGNERFRAPEALFQPSFLGMESAGVHETVHSSIMRCDIDIRKDLFANIVLSGGTTMYPGIADRMQKEITTLAPSTIKIIAPPERKYSVCIGGSILASLSTFQQMCISKQEYDESGPSIVHRKCF

SEQ ID NO: 7 - DNA, 32 bp, Artificial Sequence
  Description of Artificial Sequence  Primer
cttacaaaatgtgtcgactgaaggaaggtngc

SEQ ID NO: 8 - DNA, 32 bp, Artificial Sequence
  Description of Artificial Sequence  Primer
ccgatgcttcgatatngggatactttnagngt

SEQ ID NO: 9 - DNA, 21 bp, Artificial Sequence
  Description of Artificial Sequence  Primer
caaactatgaagacccaatcc

SEQ ID NO: 10 - DNA, 20 bp, Artificial Sequence
  Description of Artificial Sequence  Primer
atatttagttagtatgcata

SEQ ID NO: 11 - DNA, 21 bp, Artificial Sequence
  Description of Artificial Sequence  Primer
aaggaaatcgacgacatcata

SEQ ID NO: 12 - DNA, 20 bp, Artificial Sequence
  Description of Artificial Sequence  Primer
atatttagttagtatgcata

SEQ ID NO: 13 - DNA, 21 bp, Artificial Sequence
  Description of Artificial Sequence  Primer
aaggaaatcgacgacatcata

SEQ ID NO: 14 - DNA, 22 bp, Artificial Sequence
  Description of Artificial Sequence  Primer
atatggggtgtctgtaaatgtg

SEQ ID NO: 15 - DNA, 19 bp, Artificial Sequence
  Description of Artificial Sequence  Primer
atgtgcgccgattcaacaa

SEQ ID NO: 16 - DNA, 20 bp, Artificial Sequence
  Description of Artificial Sequence  Primer
aatcgggtatatattgcaca

SEQ ID NO: 17 - DNA, 19 bp, Artificial Sequence
  Description of Artificial Sequence  Primer
atgtgcgccgattcaacaa

SEQ ID NO: 18 - DNA, 20 bp, Artificial Sequence
  Description of Artificial Sequence  Primer
atatttagttagtatgcata

SEQ ID NO: 19 - DNA, 19 bp, Artificial Sequence
  Description of Artificial Sequence  Primer
atgtgcgccgattcaacaa

SEQ ID NO: 20 - DNA, 22 bp, Artificial Sequence
  Description of Artificial Sequence  Primer
atatggggtgtctgtaaatgtg

SEQ ID NO: 21 - DNA, 21 bp, Artificial Sequence
  Description of Artificial Sequence  Primer
caaactatgaagacccaatcc

SEQ ID NO: 22 - DNA, 20 bp, Artificial Sequence
  Description of Artificial Sequence  Primer
aatcgggtatatattgcaca

SEQ ID NO: 23 - DNA, 21 bp, Artificial Sequence
  Description of Artificial Sequence  Primer
aaggaaatcgacgacatcata

SEQ ID NO: 24 - DNA, 20 bp, Artificial Sequence
  Description of Artificial Sequence  Primer
aatcgggtatatattgcaca

(19) United States
(12) Patent Application Publication
(10) Pub. No.: US 2006/0242719 A1
(43) Pub. Date: Oct. 26, 2006

(54) Nucleotide sequences of shrimp beta-actin and actin promoters and their use in gentic transformation technology

(76) Inventors: Piera S. Sun, Honolulu, HI (US); Kristi L. Arakaki, Kaneohe, HI (US); Samuel Sai Ming Sun, Honolulu, HI (US)

Correspondence Address: Michael L Goldman;Nixon Peabody, Clinton Square, P O Box 31051, Rochester, NY, 14603-1051, US

(21) Appl. No.: 10/497,583

(22) PCT Filed: Dec. 4, 2002
(86) PCT No.: PCT/US02/38523

§ 371 Date: Oct. 15, 2004

Related U.S. Application Data
(60) Provisional application No. 60/336,603, filed on Dec. 4, 2001.

(51) Int. Cl. A01K 67/033; A01K 67/027; C07H 21/04; C07K 5/06; A61K 48/00

(52) U.S. Cl. 800/13; 514/44; 435/325; 536/23.2; 800/20

(57) ABSTRACT

The present invention relates to isolated actin and β-actin nucleic acid promoter molecules from shrimp; nucleic acid expression cassettes including actin and β-actin promoter molecules isolated from shrimp; and expression vectors, host cells, and transgenic animals transduced with the isolated actin and β-actin nucleic acid promoter. Also disclosed are methods for imparting to an animal resistance against a pathogen; regulating growth of an animal; increasing stress tolerance in an animal; and increasing cold tolerance in an animal that involve transforming an animal with a nucleic acid construct including the isolated actin and β-actin nucleic acid promoter molecules of the present invention. The present invention also relates to isolated nucleic acid molecules encoding for actin and β-actin proteins or polypeptides, as well as the proteins or polypeptides themselves.

This application claims the benefit of U.S. Provisional Patent Application Ser. No. 60/336,603, filed Dec. 4, 2001.

This invention was developed with government funding through NOAA/National Sea Grant Award Nos. NA36RG0507, NA86RG0041, and NA16RG2254. The U.S. Government may have certain rights.

### FIELD OF THE INVENTION

The present invention relates to nucleotide sequences of shrimp promoters which can be used in the construction of genetic transformation vectors for introducing desirable foreign DNA(s) into commercially important shellfish and crustaceans.

### BACKGROUND OF THE INVENTION

Infectious diseases among shrimp have taken a devastating toll on aquaculture production. Among the most harmful pathogens are viruses, bacteria, and protozoans, with viruses posing the greatest threat to shrimp survival rates. Bacterial and fungal infections in shrimp can usually be controlled effectively by applying available chemical treatments to shrimp populations in hatchery ponds or tanks. However, there are currently no effective chemicals or antibiotics to treat viral diseases. Other strategies used in handling shrimp disease problems include immunostimulation, vaccination, quarantining, and environmental management. These strategies are generally targeted at three elements: pathogens, host, and environment. Boosting the shrimp's natural defense system against pathogens is a non-specific approach to combating disease, yet, does not improve the shrimp's ability to cope with future outbreaks of the same disease since shrimp and other invertebrates lack a memory immune response based on antibody production. The lack of basic information about shrimp immunology is also another impediment to the development of efficient strategies for combating viral diseases via traditional methods.

Viral diseases are the most devastating problem facing shrimp aquaculture. The four major viruses, including white spot syndrome virus (WSSV), yellow head virus (YHV), Taura syndrome virus (TSV), and infectious hypodermal and hematopoietic necrosis virus (IHHNV), pose the greatest threat to penaeid shrimp farming worldwide. The IHHNV was first detected in Hawaii in 1981, causing up to 90% mortality in juvenile shrimp, Litopenaeus stylirostris (Lightner et al., “Infectious Hypodermal and Hematopoietic Necrosis, a Newly Recognized Virus Disease of Penaeid Shrimp,” J. Invert. Pathol. 42: 62-70 (1983)). This virus has since been reported to infect most Litopenaeus species (which was previously known as the Penaeus species), including the Pacific white shrimp, L. vannamei and the blue shrimp, L. stylirostris, causing tremendous economic losses worldwide (Brock, “An Overview of Diseases of Cultured Crustaceans in the Asia Pacific Region,” in Fish Health Management in Asia-Pacific. Report on a Regional Study and Workshop on Fish Disease and Fish Health Management, ADB Agriculture Department Report Series No. 1. Network of Aquaculture Centres in Asia-Pacific. Bangkok, Thailand, pp. 347-395 (1991); Flegel, “Major Viral Diseases of the Black Tiger Prawn (Penaeus Monodon) in Thailand,” in NRIA International Workshop, New approaches to viral diseases of aquatic animals, Kyoto, Japan. Jan. 21-24, 1997, National Research Institute of Aquaculture, Nansei, Mie 516-01, Japan pp. 167-189 (1997)). TSV has infected United States farms rearing Litopenaeus vannamei since 1992 and has caused more than 2 billion dollars in damage to aquaculture farms (Brock, “An Overview of Taura Syndrome, an Important Disease of Farmed Penaeus Vannamei,” in C. L. Browndy and J. S. Hopkins, (eds.), Swimming Through Troubled Water. Proceedings of the Special Section on Shrimp Farming, Baton-Rouge, La.: World Aquaculture Society pp. 84-94 (1995); Lightner et al., “Risk of Spread of Penaeid Shrimp Viruses in the Americas by the International Movement of Live and Frozen Shrimp,” Rev. Sci. Tech. 16(1):146-60 (1997)). In Hawaii, both TSV and IHHNV infections in shrimp farms have been frequently reported since 1994 (MacMillan, “Shrimp Diseases in Hawaii, USA”. UNIHI-SG-FS-96-02. University of Hawaii Sea Grant College Program, Honolulu (1996)). Controlling viral diseases clearly represents a great challenge as there are currently no effective chemicals or antibiotics to treat viral infection. The serious effects of viral disease outbreaks among cultured shrimp coupled with a decline in natural fisheries of healthy shrimp (Pullin et al., “Domestication of Crustaceans,” Asian Fisheries Sci. 11(1):59-69 (1998)), have led to a critical demand for advanced biotechnological applications.

The two major penaeid shrimp species cultured in the Americas, L. vannamei and L. Stylirostris, have differing susceptibilities to TSV and IHHNV. L. vannamei is more resistant to IHHNV, but susceptible to TSV, whereas L. stylirostris is innately resistant to TSV but highly susceptible to IHHNV (Lightner et al., “Strategies for the Control of Viral Diseases of Shrimp in the Americas,” Fish Pathology 33:165-180 (1998)). Despite the relative resistance of L. vannamei to IHHNV, runt deformity syndrome (RDS) was still observable in this shrimp species when exposed to IHHNV. Although these viral diseases may not be completely fatal, the reduced growth rate resulting from viral-induced RDS results in immense revenue losses for shrimp farmers each year.

Systematic genetic selection is known to enhance disease resistance in a number of farmed plants and animals, including fish (Gjedrem et al., “Genetic Variation in Susceptibility of Atlantic Salmon to Furunculosis,” Aquaculture 97:1-6 (1991)). However, the efficacy of breeding for disease resistance in penaeid shrimp is not well established because of the paucity of information about relevant genetic parameters, such as phenotypic and genetic variation, heritability, and genetic correlations between traits. In response to viral-disease problems facing the shrimp farming industry, the U.S. Marine Shrimp Farming Program (USMSFP), with funding from USDA/CSREES, has developed a selective breeding program to enhance disease resistance and improve growth in L vannamei (Moss et al., “Breeding for Disease Resistance in Penaeid Shrimp: Experiences From the U.S. Marine Shrimp Farming Program,” In: Proceedings of the 1^{st} Latin American Shrimp Farming Congress (D. E. Jory, ed.), Panama City, Panama, 9 pp. (1998); Argue et al., “Selective Breeding of Pacific White Shrimp (Litopenaeus Vannamei) for Growth and Resistance to Taura Syndrome Virus,” Aquaculture 204:447-460 (2002)). Although high between-family variation in response to TSV challenge was observed in all groups of shrimp tested, heritability estimates (h²) for TSV resistance were low (h²_{full-sib}=0.14). Heritability describes the percentage of phenotypic variance that is inherited in a predictable manner and is used to determine the potential response to selection (Tave, “Genetics for Fish Hatchery Managers,” 2nd ed., AVL New York, 415 pp (1993)). Estimates of h² typically are low for fitness traits, such as disease resistance, and phenotypes with h²≦0.15 are difficult to improve by selection. Although the development of TSV-resistant strains of L. vannamei have benefited shrimp farmers, breeding for TSV resistance is not a panacea to the health problems plaguing the industry. Viruses can mutate, thereby rendering selectively bred shrimp incapable of defending themselves against new strains of virus. Furthermore, TSV resistance could be negatively correlated with resistance to other pathogens. There is also the potential to produce shrimp that respond well in disease-challenge tests used in breeding programs, but perform poorly when stocked in commercial ponds.

The use of molecular biology techniques to produce pathogen-resistant strains of shrimp through genetic transformation technology is considered a highly promising strategy for control of shrimp viral disease (Mialhe et al., “Future of Biotechnology-Based Control of Disease in Marine Invertebrates,” Mol. Mar. Biol. And Biotechnol. 4(4):275-83 (1995); Bachere et al., “Transgenic Crustaceans,” World Aquaculture 28(4):51-5 (1997)). In the past decade, pathogen-resistant transgenic animals and plants have been developed (Beachy, “Virus Cross-Protection in Transgenic Plants,” in D. P. S. Verma, and R. B. Goldberg, (eds.), Plant Gene Research: Temporal and Spatial Regulation of Plant Genes, New York: Springer Verlag pp. 313-327 (1998); Kim et al., “Disease Resistance in Tobacco and Tomato Plants Transformed with the Tomato Spotted Wilt Virus Nucleocapsid Gene,” Plant Dis. 78:615-21 (1993); Sin, “Transgenic Fish,” Rev. Fish Biol. 7(4):417-41 (1997)), but use of such technology has only just begun for shrimp research. While methods for detecting viral disease in shrimp, including polymerase chain reaction (Dhar et al., “Detection and Quantification of Infectious Hypodermal and Hematopoietic Necrosis Virus (IHHNV) and White Spot Virus (WSV) of Shrimp by Real-Time Quantitative PCR and SYBR Chemistry,” J. Clin. Microbiol. 39:2835-2845 (2001); Tang et al., “Detection and Quantification of Infectious Hypodermal and Hematopoietic Necrosis Virus in Penaeid Shrimp by Real-Time PCR,” Dis. Aquat. Org. 44(2):79-85 (2001)), light microscopy, and transmission electron microscopy (Nunan et al., “Reverse Transcription Polymerase Chain Reaction (RT-PCR) Used for the Detection of Taura Syndrome Virus (TSV) in Experimentally Infected Shrimp,” Dis. Aquatic. Org. 34:87-91 (1998); Goarant et al., “Arbitrarily Primed PCR to Type Vibrio Spp. Pathogenic for Shrimp,” Appl. Environ. Microbiol. 65(3):1145-1151 (1999); Chen et al., “Establishment of Cell Culture Systems from Penaeid Shrimp and Their Susceptibility to White Spot Disease and Yellow Head Viruses,” Meth, in Cell Sci. 21:199-206 (1999); Toullec, “Crustacean Primary Cell Culture: a Technical Approach,” Meth. in Cell Sci. 21:193-8 (1999); Sukhumsirichart et al., “Characterization and PCR Detection of Hepatopancreatic Parvovirus (HPV) from Penaeus Monodon in Thailand,” Dis. Aquat. Org. 38:1-10 (1999), are widely used, methods for controlling viral disease in shrimp are still in development. The first studies on genetic transformation of marine molluscs and shrimp were initiated in 1988 in France at IFREMER, in the United States at the University of Maryland Biotechnology Institute, and in Australia at CSIRO. A few studies on the introduction of foreign DNA into shrimp embryos via transfection methods have obtained preliminary data demonstrating transient expression of a reporter gene by heterologous promoters (Gendreau et al., “Transient Expression of a Luciferase Reporter Gene After Ballistic Introduction Into Artemia franciscana (Crustacea) Embryos,” Aquaculture 133:199-205 (1995)). Recent advances in gene transfer technology such as these hold immense potential for developing transgenic shrimp harboring genes that convey viral disease resistance or enhance shrimp growth rates. Gene transfer technology thus represents a practical alternative to the lengthy and expensive selective breeding process (Wolfus et al., “Application of the Microsatellite Technique for Analyzing Genetic Diversity in Shrimp Breeding Programs,” Aquaculture 152:35-47 (1997)), and provides a powerful tool for revolutionizing not only shrimp aquaculture, but also livestock husbandry in general.

Construction of an effective expression vector is an important step toward implementing the genetic transformation process in animals. The expression vector is generally composed of three elements: a promoter, a target gene, and a region having transcriptional termination signals. Among these three components, a suitable promoter is the most important element for a successful gene transformation system. The promoter determines where, when, and under what conditions the target gene should be turned on.

A suitable promoter that is appropriate for aquaculture and acceptable to consumers should ideally be derived from marine origin and should not pose any potential health hazards. Several fish gene promoters have been successfully isolated and used to drive foreign gene expression (Jankowski et al., “The GC Box as a Silencer,” Biosci. Rep. 7:955-63 (1987); Zafarullah et al., “Structure of the Rainbow Trout Metallothionein B Gene and Characterization of its Metal-Responsive Region,” Mol. Cell. Biol. 8:4469-76 (1988); Liu et al., “Development of Expression Vectors for Transgenic Fish,” Bio/Technoloy 8:1268-1272 (1990b); Gong et al., “Functional Analysis and Temporal Expression of Promoter Regions From Fish Antifreeze Protein Genes in Transgenic Japanese Medaka Embryos,” Mol. Mar. Biol. Biotechnol. 1(1):64-72 (1991); Du et al., “Growth Enhancement in Transgenic Atlantic Salmon by the Use of Fish Antifreeze/Growth Hormone Chimeric Gene Constructs,” Biotechnology 10:176-81 (1992); Gong et al., “Transgenic Fish in Aquaculture and Developmental Biology,” Current Topic in Develop. Biol. 30:175-213 (1995); Chen et al., “Transgenic Fish and Aquaculture,” Biotechnol. Apl. 13(1):50 (1996); Chan et al., “PCR Cloning and Expression of the Molt-Inhibiting Hormone Gene for the Crab (Charybdis feriatus),” Gene 224:23-33 (1998); Gong, “Zebrafish Expressed Sequence Tags and Their Applications,” Meth. Cell Biol. (zebrafish volume) 60:213-233 (1998); Ju et al., “Faithful Expression of Green Fluorescent Protein (GFP) in Transgenic Zebrafish Embryos Under Control of Zebrafish Gene Promoters,” Dev. Genet. 25(2):158-67 (1999); Yoshizaki et al., “Germ Cell-Specific Expression of Green Fluorescent Protein in Transgenic Rainbow Trout Under Control of the Rainbow Trout Vasa-Like Gene Promoter,” Int. J. Dev. Biol. 44(3):323-6 (2000)). Other promoters used to date in transgenic marine fish include mouse metallothionein (McEvoy et al., “The Expression of a Foreign Gene in Salmon Embryos,” Aquaculture 68:27-37 (1988); Rahman et al., “Fish Transgene Expression by Direct Injection Into Fish Muscle,” Mol. Mar. Biol. Biotechnol. 1:286-289 (1992)), heat shock promoters (Bayer et al., “A Transgene Containing lacZ is Expressed in Primary Sensory Neurons in Zebrafish,” Development 115:421-446 (1992); Krone, “Several Unique Hsp 90 Genes are Expressed During Embryonic Development of Zebrafish,” Presented at Symposium on Advances in Molecular Endocrinology of Fish, May 23-25, Toronto, Canada (1993)), chicken β-actin promoter (Lu et al., “Integration and Germline Transmission of Human Growth Hormone Gene in Medaka (Oryzias latipes),” presented at Second International Marine Biotechnology Conference, 1991, Baltimore, Md. (1991); Inoue et al., “Introduction, Expression, and Growth-Enhancing Effect of Rainbow Trout Growth Hormone cDNA Fused to an Avian Chimeric Promoter in Rainbow Fry,” J. Mar. Biotechnol. 1:131-4 (1993)), carp β-actin promoter (Liu et al., “Functional Analysis of Elements Affecting Expression of the β∃-Actin Gene of Carp,” Mol. Cell Biol. 10:3432-3440 (1990); Rahman et al., “Fish Transgene Expression by Direct Injection Into Fish Muscle,” Mol. Mar. Biol. Biotechnol. 1:286-289 (1992)), the antifreeze protein promoter from the ocean pout (Macrozoarces americanus) (Gong et al., “Functional Analysis and Temporal Expression of Promoter Regions From Fish Antifreeze Protein Genes in Transgenic Japanese Medaka Embryos,” Mol. Mar. Biol. Biotechnol. 1(1):64-72 (1991); Hew et al., “Antifreeze Protein Gene Transfer in Atlantic Salmon,” Presented at Second International Marine Biotechnology Conference, 1991, Baltimore, Md. (1991); Du et al., “Growth Enhancement in Transgenic Atlantic Salmon by the Use of Fish Antifreeze/Growth Hormone Chimeric Gene Constructs,” Biotechnology 10:176-81 (1992)), and the histone promoter from the trout (Muller et al., “Introducing Foreign Genes Into Fish Eggs With Electroporated Sperm as a Carrier,” Mol. Mar. Biol. Biotechnol. 1:276-281 (1992)). Unfortunately, these promoters have disadvantages, including inconsistent transgenic expression, potential toxicity due to their viral origin, and association with metabolic poisons and/or tumor-inducing sequences, all of which will present major stumbling blocks toward attaining FDA approval for the commercial use of transgenic animals. However, isolation and use of promoter genes from crustacean shrimp has not been reported. Thus, the tremendous potential presented by gene transfer technology has not yet been realized in shrimp aquaculture due to the lack of a constitutive, non-inducible, and non-developmentally regulated promoter to efficiently drive the expression of heterologous genes in shrimp and other marine animals.

The present invention is directed to overcoming these and other deficiencies in the art.

### SUMMARY OF THE INVENTION

The present invention relates to an isolated β-actin nucleic acid promoter molecule from shrimp having a nucleotide sequence comprising one or more (GC)-rich regions (i.e., regions rich in G and C).

The present invention also relates to an isolated nucleic acid molecule encoding β-actin from shrimp, where the nucleic acid molecule either 1) has a nucleotide sequence of SEQ ID NO: 2; or 2) encodes a protein having SEQ ID NO: 3.

The present invention also relates to an isolated shrimp β-actin having an amino acid sequence of SEQ ID NO: 3.

The present invention also relates to expression vectors, host cells, and transgenic animals transduced with the isolated β-actin nucleic acid promoter molecule from shrimp, and methods for imparting to an animal resistance against a pathogen, regulating growth of an animal, and increasing stress tolerance in an animal, that involve transforming an animal with a nucleic acid construct including the isolated β-actin nucleic acid promoter molecule from shrimp having a nucleotide sequence comprising one or more (GC)-rich regions.

The present invention also relates to an isolated actin nucleic acid promoter molecule from shrimp having a nucleotide sequence comprising (CATA)-rich repeats and (CACA)-rich repeats.

Another aspect of the present invention is an isolated nucleic acid molecule encoding actin from shrimp, wherein the nucleic acid molecule either 1) has a nucleotide sequence of SEQ ID NO: 5; or 2) encodes a protein having SEQ ID NO: 6.

The present invention also relates to an isolated shrimp actin having an amino acid sequence of SEQ ID NO: 6.

The present invention also relates to expression vectors, host cells, and transgenic animal transduced with the isolated actin nucleic acid promoter molecule from shrimp, and methods of imparting to an animal resistance against a pathogen, regulating growth of an animal, and increasing stress tolerance in an animal, that involve transforming an animal with a nucleic acid construct including the isolated actin nucleic acid promoter molecule from shrimp having a nucleotide sequence comprising (CATA)-rich repeats and (CACA)-rich repeats.

Transgenic strains of animals with new and desirable genetic traits may offer great benefits in marine aquaculture. For example, control of infectious diseases and acceleration of growth rate, two of the most important challenges facing commercial shrimp aquaculture today, may be answered by the application of recombinant DNA technology to these problems. However, genetic engineering of shrimp and other crustaceans requires a suitable promoter that, ideally, is constitutive, non-inducible, non-developmentally regulated, and derived from marine origin so as not to pose any potential health hazards. The present invention provides such promoters, and uses advanced recombinant DNA technology to produce transgenic marine animals in which one or more desirable DNA sequences can be introduced.

### BRIEF DESCRIPTION OF THE DRAWINGS

FIG. 1 is a schematic diagram of the shrimp β-actin gene and its promoter. Numbers represent nucleotide base pairs. UR: untranslated region, ATG: translation start site, SP: signal peptide, MP: mature peptide, TAA: translation stop site. Regulatory regions (TATA, CAAT, CArG boxes and GC-rich regions) are present 100-1100 bp upstream from the translation start site. Drawing is not to scale.

FIG. 2 is a schematic diagram of the shrimp skeletal muscle actin (“actin”) gene and its promoter. Numbers represent nucleotide base pairs. UR: untranslated region, ATG: translation start site, SP: signal peptide, MP: mature peptide, TAA: translation stop site. Regulatory regions (TATA and CAAT boxes, CACA-rich and CATA-rich regions) are present approximately 500-1350 bp upstream from the translation start site. Drawing is not to scale.

FIGS. 3A-B show a comparison of marker gene expression efficiency in shrimp muscle. Marker EGFP is shown in FIG. 3A, compared to DsRed, shown in FIG. 3B.

FIG. 4 is an ethidium bromide/agarose gel (2%) electrophoresis analysis of RT-PCR products encoding the TSV-CP with four sets of gene-specific primers. DNA fragments of 471-bp, 574-bp, 1020-bp, 1123-bp, 573-bp and 770-bp are shown in Lanes 2, 3, 4, 5, 6, and 7, respectively. A DNA size marker is shown in Lane 1.

FIG. 5 is an ethidium bromide/agarose gel (2%) electrophoresis analysis of RT-PCR products. Lane 1:100 bp DNA molecular marker; Lane 2: primer pair #1; Lane 3: primer pair #2; Lane 4: primer pair #3; Lane 5: primer pair #4; Lane 6: primer pair #5; Lane 7: primer pair #6; Lane 8: primer pair #7; Lane 9: primer pair #8; Lane 10: cloned IHHNV DNA band generated with primer pair #8 that includes the full length sequence of the IHHNV-coat protein; and Lane 11: 100 bp DNA molecular marker.

FIGS. 6A-C are expression vectors consisting of the chimeric shrimp β-actin promoter, sense or antisense TSV-CP target gene, and reporter β-galactosidase gene (or EGFP gene). FIG. 6A is the pβ-ActinP2-β-Gal vector construct. FIG. 6B is pβ-ActinP2-TSV-CP-AS (471 bp) with the TSV-CP target gene in the antisense orientation vector. FIG. 6C shows pβ-ActinP2-TSV-CP-S (471 bp), constructed with the TSV-CP target gene in the sense orientation.

FIG. 7 is the plasmid map of vector pβ-ActinP2-TSV-CP-S.

FIG. 8 is the plasmid map of vector pβ-ActinP2-TSV-CP-AS.

FIG. 9 is the plasmid map of vector pβ-ActinP2-β-Gal.

FIG. 10 is the plasmid map of vector pβ-ActinP2-P26.

FIG. 11 is the plasmid map of vector pβ-ActinP3-EGFP.

FIG. 12 is the plasmid map of vector pβ-ActinP1-EGFP.

FIG. 13 is a graph comparing the efficiency of the shrimp, chicken, and human cytomegalovirus (CMV) promoters in expressing EGFP in shrimp.

FIGS. 14A-B are graphs comparing the efficiency of the shrimp pβ-ActinP2-β-Gal vector of against control vectors using microinjection and electroporation. FIG. 14A shows the efficiency of β-ActinP2 promoter in β-Gal expression at different pulse lengths of electroporation of A. franciscana embryos. FIG. 14B shows efficiency of β-ActinP2 promoter compared to the CMV promoter in β-Gal expression in microinjected A. franciscana embryos.

FIG. 15 is a graph comparing hatching of L. vannamei shrimp embryos following transfection with various ratios of plasmid DNA/SuperFect

FIG. 16 is an ethidium bromide/agarose gel (2%) electrophoresis analysis of RT-PCR detection of target gene, TSV-CP (antisense), expression in electroporated L. vannamei. Lane 1: molecular marker. Lane 2: experimental shrimp. Lane 3: shrimp electroporated with PBS. Lane 4: positive control.

FIG. 17 is an ethidium bromide/agarose gel (2%) electrophoresis analysis of RT-PCR detection of target gene, TSV-CP (sense), expression in microinjected L. vannamei. Lane 1: molecular marker. Lane 2: experimental shrimp. Lane 3: negative control shrimp. Lane 4: positive control.

### DETAILED DESCRIPTION OF THE INVENTION

The present invention relates to an isolated β-actin nucleic acid promoter molecule from shrimp having a nucleotide sequence comprising GC-rich regions. This promoter, isolated and cloned from the Pacific white shrimp, Litopenaeus vannamei, has a nucleotide sequence of SEQ ID NO: 1, as follows:

| | |
|---|---|
| aaaaggatct aggtgaagat cctttttgat aatctcatga | 60 |
| ccaaaatccc ttaacgtgag | |
| ttttcgttcc actgagcgtc agaccccgta gaaaagatca | 120 |
| aaggatcttc ttgagatcct | |
| ttttttctgc gcgtaatctg ctgcttgcaa acaaaaaaac | 180 |
| caccgctacc agcggtggtt | |
| gtttgccgga tcaagagcta ccaactcttt ttccgaagta | 240 |
| actggcttca gcagagcgca | |
| gataccaaat actgttcttc tagtgtagcc gtagttaggc | 300 |
| caccacttca agaactctgt | |
| agcaccgcct acatacctcg ctctgctaat cctgttacca | 360 |
| gtggctgctg ccagtggcga | |
| taagtcgtgt cttaccgggt tggactcaag acgatagtta | 420 |
| ccggataagg cgcagcggtc | |
| gggctgaacg gggggttcgt gcacacagcc cagcttggag | 480 |
| cgaacgacct acaccgaact | |
| gagataccta cagcgtgagc tatgagaaag cgccacgctt | 540 |
| cccgaaggga gaaaggcgga | |
| caggtatccg gtaagcggca gggtcggaac aggagagcgc | 600 |
| acgagggagc ttccaggggg | |
| aaacgcctgg tatctttata gtctgtcggg tttcgccacc | 660 |
| tctgacttga gcgtcgattt | |
| ttgtgatgct cgtcaggggg gcggagccta tggaaaaacg | 720 |
| ccagcaacgc ggccttttta | |
| cggttcctgg ccttttgctg gccttttgct cacatgttct | 780 |
| ttcctgcgtt atcccctgat | |
| tctgtggata accgtattac cgcctttgag tgagctgata | 840 |
| ccgctcgccg cagccgaacg | |
| accgagcgca gcgagtcagt gagcgaggaa gcggaagagc | 900 |
| gcccaatacg caaactcgcc | |
| tctccccgcg cgttggccga ttcattaatg cagctggcac | 960 |
| gacaggtttc ccaactggaa | |
| agcgggcagt gagcgcaacc caattaatgt gagttagctc | 1020 |
| actcattagg caccccaggc | |
| tttacacttt atgcttccgg ctcgtatgtt gtgtggaatt | 1080 |
| gtgagcggat aacaatttca | |
| cacaggaaac agctatgacc atgattacgc caagctcgaa | 1140 |
| attaaccctc actaaaggga | |
| acaaaagctg gagctcccca ccgcggtggc ggccgctcta | 1200 |
| gaactagtgg atcccccggg | |
| ctgcaggaat ttggcacgag ctgccgaatt cggcacgagc | 1260 |
| ccgagaggaa gcagcacgta | |
| cgctcgtccg cccttttagt aaaacaacaa caacaag | 1297 |

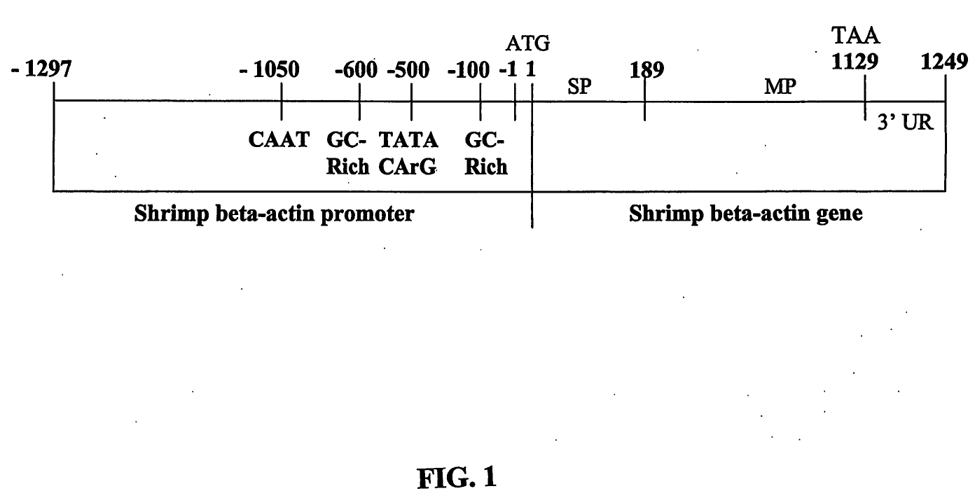

This β-actin promoter of the present invention is a constitutive, non-inducible, and non-developmentally regulated promoter. It is suitable for inducing expression of a protein encoded by a nucleic acid molecule operably associated with the promoter molecule in an expression vector. As shown in FIG. 1, the shrimp β-actin promoter of the present invention contains regulatory elements including a TATA box, CarG box, and CAAT box. It is interesting to note that the TATA box is located between two highly GC-rich regions. While the TATA and CAAT boxes are conserved among nucleic acid promoter molecules, the GC-rich regions located at 676-688 and 1161-1176 in the shrimp β-actin promoter of the present invention are not common, and appear to be characteristic of this particular promoter.

In the shrimp, the β-actin promoter contains a complex array of cis-acting regulatory elements required for accurate and efficient initiation of transcription and for controlling expression of the β-actin gene. Transcripts of the shrimp β-actin gene are found in most of the major shrimp organs including the eyestalk, brain, heart, and hepatopancreas, suggesting that the shrimp β-actin is a cytoplasmic form of actin whose expression is constitutive, non-developmentally regulated, and non-inducible, and thus should remain constant throughout the lifespan of the shrimp.

The present invention also relates to an isolated nucleic acid molecule encoding β-actin from the Pacific white shrimp, Litopenaeus vannamei, where the nucleic acid molecule has a nucleotide sequence of SEQ ID NO: 2, as follows:

| | |
|---|---|
| atgtgtgacg acgaagtagc cgccctggtt gtagacaatg | 60 |
| gctccggcat gtgcaaggcc | |
| ggcttcgctg gtgacgatgc accacgagct gtgttcccct | 120 |
| ccatcgtcgg ccgaccccgt | |
| catcagggtg tgatggtcgg catgggccag aaggactcgt | 180 |
| acgtcggcga cgaggcccag | |
| agcaagcgag gtatcctcac cctgaaatac cccatcgagc | 240 |
| acggcatcgt caccaactgg | |
| gacgacatgg agaagatctg gcatcacact ttctacaacg | 300 |
| agctccgcgt ggcccccgag | |
| gagcaccccg tcctgctgac cgaggctccc ctcaacccca | 360 |
| aggctaaccg cgagaagatg | |
| acacagatca tgttcgagac cttcaacacc cccgccatgt | 420 |
| acgtggccat ccaggccgtg | |
| ctgtccctgt acgcctccgg ccgtaccacc ggtatcgtgc | 480 |
| tcgactccgg cgacggcgtg | |
| tcccacaccg tgcccatcta cgagggatat gccctgcccc | 540 |
| acgccatcct gcgtctggac | |
| ttggccggcc gcgacctcac agactacctg atgaagatcc | 600 |
| tgacggagcg tggctacacc | |
| ttcacgacca ccgccgagcg agaaatcgtt cgtgacatca | 660 |
| aggagaaact gtgctacgtg | |
| gccctggact tcgagcagga gatgaccacc gctgcttcct | 720 |
| cctcctcgct ggagaagtcc | |
| tacgagctcc ctgacggcca ggtgatcacc atcggcaacg | 780 |
| agaggttccg ctgccccgag | |
| gccctgttcc agccctcatt cctgggcatg gaatcctgcg | 840 |
| gcatccacga gaccacctac | |
| aactccatca tgaagtgcga cgtggacatc cgtaaggacc | 900 |
| tgtacgccaa caccgtgctg | |
| tccggaggca ccaccatgta ccctggcatc gccgacagga | 960 |
| tgcagaagga aatcactgcc | |
| ctcgctccct ccaccatgaa gatcaagatc atcgccccac | 1020 |
| ccgagcgcaa gtactccgtg | |
| tggatcggcg gctccatcct ggcctcgctc tccaccttcc | 1080 |
| agcagatgtg gatcagcaag | |
| caggagtacg acgagtctgg accatcaatt gttcacagga | 1140 |
| agtgcttcta attaacaaaa | |
| tgtacatgat ataggctaca ctttttacat ttaattattc | 1200 |
| cattaggata aggattatgt | |
| tatttaaaag gaataaattc atttctacaa aaaaaaaaaa | 1249 |
| aaaaaaaaa | |

The nucleic acid molecule having a nucleotide sequence of SEQ ID NO: 2 encodes a β-actin polypeptide or protein of the present invention isolated from Litopenaeus vannamei, which has a deduced amino acid sequence of SEQ ID NO: 3, as follows:

```
Met Cys Asp Asp Glu Val Ala Ala Leu Val Val Asp
    1 5 10
Asn Gly Ser Gly
                  15
Met Cys Lys Ala Gly Phe Ala Gly Asp Asp Ala Pro
                          20 25
Arg Ala Val Phe
          30
Pro Ser Ile Val Gly Arg Pro Arg His Gln Gly Val
                  35 40
Met Val Gly Met
  45
Gly Gln Lys Asp Ser Tyr Val Gly Asp Glu Ala Gln
          50 55 60
Ser Lys Arg Gly
Ile Leu Thr Leu Lys Tyr Pro Ile Glu His Gly Ile
  65 70 75
Val Thr Asn Trp
                          80
Asp Asp Met Glu Lys Ile Trp His His Thr Phe Tyr
                                  85 90
Asn Glu Leu Arg
                  95
Val Ala Pro Glu Glu His Pro Val Leu Leu Thr Glu
                        100 105
Ala Pro Leu Asn
        110
Pro Lys Ala Asn Arg Glu Lys Met Thr Gln Ile Met
                115 120
Phe Glu Thr Phe
125
Asn Thr Pro Ala Met Tyr Val Ala Ile Gln Ala Val
        130 135 140
Leu Ser Leu Tyr
Ala Ser Gly Arg Thr Thr Gly Ile Val Leu Asp Ser
145 150 155
Gly Asp Gly Val
                        160
Ser His Thr Val Pro Ile Tyr Glu Gly Tyr Ala Leu
                                165 170
Pro His Ala Ile
                175
Leu Arg Leu Asp Leu Ala Gly Arg Asp Leu Thr Asp
                        180 185
Tyr Leu Met Lys
        190
Ile Leu Thr Glu Arg Gly Tyr Thr Phe Thr Thr Thr
                195 200
Ala Glu Arg Glu
205
Ile Val Arg Asp Ile Lys Glu Lys Leu Cys Tyr Val
        210 215 220
Ala Leu Asp Phe
Glu Gln Glu Met Thr Thr Ala Ala Ser Ser Ser Ser
225 230 235
Leu Glu Lys Ser
                        240
Tyr Glu Leu Pro Asp Gly Gln Val Ile Thr Ile Gly
                                245 250
Asn Glu Arg Phe
                255
Arg Cys Pro Glu Ala Leu Phe Gln Pro Ser Phe Leu
                        260 265
Gly Met Glu Ser
        270
Cys Gly Ile His Glu Thr Thr Tyr Asn Ser Ile Met
                275 280
Lys Cys Asp Val
285
Asp Ile Arg Lys Asp Leu Tyr Ala Asn Thr Val Leu
        290 295 300
Ser Gly Gly Thr
Thr Met Tyr Pro Gly Ile Ala Asp Arg Met Gln Lys
305 310 315
Glu Ile Thr Ala
                        320
Leu Ala Pro Ser Thr Met Lys Ile Lys Ile Ile Ala
                                325 330
Pro Pro Glu Arg
                335
Lys Tyr Ser Val Trp Ile Gly Gly Ser Ile Leu Ala
                        340 345
Ser Leu Ser Thr
        350
Phe Gln Gln Met Trp Ile Ser Lys Gln Glu Tyr Asp
                355 360
Glu Ser Gly Pro
365
Ser Ile Val His Arg Lys Cys Phe
        370 375
```

The deduced polypeptide of the shrimp beta-actin consists of a 63-amino acid signal peptide and a 313-amino acid mature polypeptide. This shrimp β-actin exhibits 99% amino acid homology with rainbow trout (Oncorhynchus mykiss) β-actin, 98% homology with fruit fly (Drosophila melanogaster) β-actin5C, and 98% homology with chicken (Gallus gallus) β-actin.

The present invention also relates to an isolated nucleic acid promoter molecule from shrimp skeletal muscle actin having a nucleotide sequence CATA-rich repeats and CACA-rich repeats. This actin promoter, isolated and cloned from the Pacific white shrimp, Litopenaeus vannamei, has a nucleotide sequence of SEQ ID NO: 4, as follows:

| | |
|---|---|
| ggactcgatc tggccatccc tcttggctcg atgtcgcatt | 60 |
| cttgggtagt agcgtagggc | |
| tagttcgcgg taagtctgta taaaagggtg cacctgcctc | 120 |
| taaccaggta gtcgtgtcaa | |
| ggctcaaacc cgggtaagtg caacgtgaca caaagcgtgg | 180 |
| ctcaggtgcg gcgaaaaggt | |
| gcaagtgtgt gttgaggtga aaataaaagt ggtgtggagg | 240 |
| attaagaagg ttgctggacg | |
| tcacttatca ttgtgtgaac atttagtatg gattataaat | 300 |
| cagttgcttt catatgtatg | |
| tgtatctaga agtacgtgtg gtttatgcca ttgtgaaaga | 360 |
| ggtttaagag aaacagaaag | |
| taatggagat gtaattactt ttagttgctg aatattgcaa | 420 |
| acggttactt gagcacgagg | |
| acatctttat acattgtata atcattaaca tccatttcag | 480 |
| tggttaaaat aataattata | |
| atagataaag gcagttataa tagacattgt ctattaatga | 540 |
| gagtcaatca gatatgctaa | |
| aaggatatag gctatgcgta attatattgc ttacggaaaa | 600 |
| ctatttgaaa gattcacaca | |
| aaagataata tctgaactat attggattat attgaatgtt | 660 |
| gaccccacat ttatatatat | |
| atatatgtat atgtatatat atgtatttat atacataaat | 720 |
| atacacatat tcatgtatat | |
| atatatgtat atatacatat atatttatat atatgcatat | 780 |
| atatatgtat atatatatat | |
| atacatatgc atattcatat atatgtatat atatacatat | 840 |
| acatatatat gtatatatat | |
| gcataaatat atgtatatga atatatatat atatatacat | 900 |
| acatacatac atatatatat | |
| atatatatat atatatatat atatatatat atatatacac | 960 |
| acacacacat gcacgcacat | |
| atacacacac acaaacacac acacatatat atgtaaatgc | 1020 |
| atacatatgt gtacacggac | |
| acatatatac acatatatac atacacacac aacagataca | 1080 |
| tatgcatata catacataca | |
| catatatctc tctctatata catatatatg tatatatgtg | 1140 |
| tatatactta tacataaaaa | |
| cacacatttg cgcacacaca tacacaatat acgacgcata | 1200 |
| catatgcata taaacacaca | |
| caaacacaca tatatccata tctgtctatc aatgtacaca | 1260 |
| tacacatata tctatatcta | |
| tctattaatg cacacacaca gagacacaga cacacacatt | 1320 |
| tatgtataat atatatatat | |
| atatatatat atatatatat atatatatat atacatatat | 1380 |
| atttatgtat atacatacag | |
| tatatacaca catacacaca aacacattta tctatatatc | 1440 |
| tttatctatc tatctgccta | |
| tcaatcaatc aatctatcta tctatctatc tataatatct | 1500 |
| atctatctat ctatctatct | |
| atctatctat ctatatatat gtatgtatgt atgtatacac | 1560 |
| acacacacat aagcacacac | |
| acacacatac acacacacac acacatatat acacacacaa | 1620 |
| tctgcctatc aatcaatcag | |
| tatatatgta cacacacaca tatatatgcg cagactcata | 1680 |
| attgcacata tacacacaca | |
| catatatata cagtatatat atatatatat atatatatat | 1740 |
| atatatatat atatatatat | |
| atgtatatat atatatactc atatatacat acacacacat | 1800 |
| acacgcacac acacacacac | |
| acatatacac atgtaagttc acaggtacac agtatatata | 1860 |
| cataaatttt ttctctgtct | |
| ttatctttat gtattaacaa atgtgtatgt atatatatcg | 1920 |
| tatatgtata tatggtatat | |
| cagattcata catatgggta tatctataaa taattaatca | 1980 |
| ctcgtgtttc gtagaatgtc | |
| tgtaaatagg cgcaagcatg cacactcact tttaaacgct | 2040 |
| tgtgtacacc tgcacgtatg | |
| cacaaaaggt gtatgtaatt ttacataaat actacaacag | 2100 |
| cacatacata tacatgataa | |
| tgtttgggta atctcagtaa tacaaaggta tctatgaaca | 2160 |
| ttaatctcat tttcatttta | |
| tctcaacagt gtcctctgac aactcaccta tatcacaatc | 2200 |

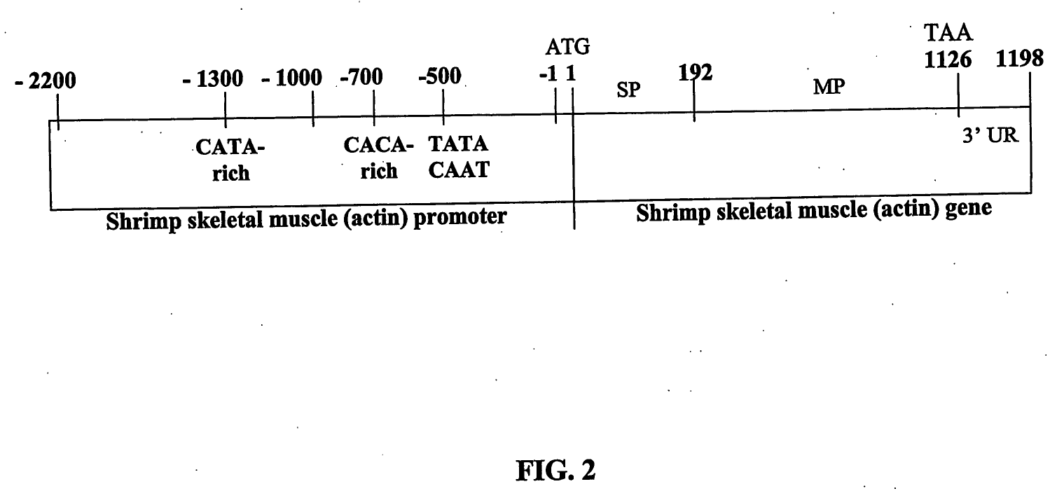

This promoter is suitable for inducing expression of a protein encoded by a nucleic acid molecule operably associated with the promoter molecule in an expression vector. The shrimp actin promoter of the present invention, as shown in FIG. 2, contains the expected promoter-associated TATA and CAAT boxes approximately 500 base pairs upstream from the translation start site. A unique characteristic of this promoter are CACA-rich and CATA-rich regions located upstream from the TATA and CAAT boxes at 878-893, 1071-1078, 1538-1549, 1554-1567, and 1570-1585.

Another aspect of the present invention is an isolated nucleic acid molecule encoding a skeletal muscle actin protein or polypeptide from shrimp, wherein the nucleic acid molecule has a nucleotide sequence of SEQ ID NO: 5, as follows:

| | |
|---|---|
| atgtgtgacg acgaagactc gtgtgcgctc gtgtgcgaca | 60 |
| atggctccgg tatggtcaag | |
| gccggattcg caggagacga cgcccctcgc gccgtcttcc | 120 |
| catccatcgt tggtcgtgct | |
| cgtcaccagg gtgtgatggt cggtatgggt cagaaggacg | 180 |
| cctacgttgg tgatgaggcc | |
| cagagcaaac gtggtatcct caccctcaag taccccattg | 240 |
| agcacggtat catcaccaac | |
| tgggatgaca tggagaagat ctggtaccac accttctaca | 300 |
| atgagctccg tgttgcccct | |
| gaagagtccc ccacacttct cactgaggct cccctcaacc | 360 |
| ccaaggccaa ccgtgagaag | |
| atgactcaga tcatgttcga gtccttcagt ctccctgcca | 420 |
| tgtatgtgac catccaggct | |
| gtgctttctc tgtacgcctc tggtcgtacc actggtcagg | 480 |
| tttgcgactc tggtgatggt | |
| gtgacccaca tggtccccgt ttatgaaggt ttcgctcttc | 540 |
| ctcatgctat ccttcgtgtt | |
| gatttagctg gtcgtgatct taccaactac ctgatgaaga | 600 |
| tcatgactga gcgtggctac | |
| tccttcacta ccaccgctga acgtgaaatc gtccgtgaca | 660 |
| tcaaggagaa gctttgctac | |
| atcgcccttg atttcgaagg tgagatgaac gtcgctgctg | 720 |
| cttcctcctc cttggacaag | |
| tcctacgaac ttcctgacgg tcaggtcatc accatcggta | 780 |
| acgagcgttt ccgtgctccc | |
| gaggctctgt tccagccttc cttccttggt atggaatctg | 840 |
| ctggtgttca ggaaaccgtg | |
| cacagctcta tcatgaggtg cgacattgac atcaggaagg | 900 |
| atctgttcgc caatattgtg | |
| ctctctggtg gtactactat gtaccctggt attgctgacc | 960 |
| gcatgcagaa ggaaatcaca | |
| accttggctc cttccaccat caagatcatc gccccacccg | 1020 |
| agcgcaagta ctccgtgtgg | |
| atcggcggct ccatcctggc ctcgctctcc accttccagc | 1080 |
| agatgtggat cagcaagcag | |
| gagtacgacg agtctggacc atcaattgtt cacaggaagt | 1140 |
| gcttctaaat gtaggagact | |
| gaaaactttt attacagttg ataataaaat ccaaaagcaa | 1198 |
| aaaaaaaaaa aaaaaaaa | |

The nucleic acid molecule having a nucleotide sequence of SEQ ID NO: 5 encodes an actin protein or polypeptide of the present invention isolated from the Pacific white shrimp, Litopenaeus vannamei, which has a deduced amino acid sequence of SEQ ID NO: 6, as follows

```
Met Cys Asp Asp Glu Asp Ser Cys Ala Leu Val Cys
    1 5 10
Asp Asn Gly Ser
                  15
Gly Met Val Lys Gly Gly Phe Ala Gly Asp Asp Ala
                          20 25
Pro Arg Ala Val
          30
Phe Pro Ser Ile Val Gly Arg Ala Arg His Gln Gly
                  35 40
Val Met Val Gly
  45
Met Gly Gln Lys Asp Ala Tyr Val Gly Asp Glu Ala
          50 55 60
Gln Ser Lys Arg
Gly Ile Leu Thr Leu Lys Tyr Pro Ile Glu His Gly
  65 70 75
Ile Ile Thr Asn
                          80
Trp Asp Asp Met Glu Lys Ile Trp Tyr His Thr Phe
                                  85 90
Tyr Asn Glu Leu
                  95
Arg Val Ala Pro Glu Glu Ser Pro Thr Leu Leu Thr
                        100 105
Glu Ala Pro Leu
        110
Asn Pro Lys Ala Asn Arg Glu Lys Met Thr Gln Ile
                115 120
Met Phe Glu Ser
125
Phe Ser Leu Pro Ala Met Tyr Val Thr Ile Gln Ala
        130 135 140
Val Leu Set Leu
Tyr Ala Ser Gly Arg Thr Thr Gly His Val Cys Asp
145 150 155
Ser Gly Asp Gly
                        160
Val Thr His Met Val Pro Val Tyr Glu Gly Ile Ala
                                165 170
Arg Pro His Ala
                175
Ile Leu Arg Val Asp Leu Ala Gly Arg Asp Leu Thr
                        180 185
Asn Tyr Leu Net
        190
Lys Ile Met Thr Glu Arg Gly Tyr Ser Phe Thr Thr
                195 200
Thr Ala Glu Arg
205
Glu Ile Val Arg Asp Ile Lys Glu Lys Leu Cys Tyr
        210 215 220
Ile Ala Leu Asp
Phe Glu Gly Glu Met Asn Val Ala Ala Ala Ser Ser
225 230 235
Ser Leu Asp Lys
                        240
Ser Tyr Glu Leu Pro Asp Gly Gln Val Ile Thr Ile
                                245 250
Gly Asn Glu Arg
                255
Phe Arg Ala Pro Glu Ala Leu Phe Gln Pro Ser Phe
                        260 265
Leu Gly Met Glu
        270
Ser Ala Gly Val His Glu Thr Val His Ser Ser Ile
                275 280
Met Arg Cys Asp
285
Ile Asp Ile Arg Lys Asp Leu Phe Ala Asn Ile Val
        290 295 300
Leu Ser Gly Gly
Thr Thr Met Tyr Pro Gly Ile Ala Asp Arg Met Gln
305 310 315
Lys Glu Ile Thr
                        320
Thr Leu Ala Pro Ser Thr Ile Lys Ile Ile Ala Pro
                                325 330
Pro Glu Arg Lys
                335
Tyr Ser Val Cys Ile Gly Gly Ser Ile Leu Ala Ser
                        340 345
Leu Ser Thr Phe
        350
Gln Gln Met Cys Ile Ser Lys Gln Glu Tyr Asp Glu
                355 360
Ser Gly Pro Ser
365
Ile Val His Arg Lys Cys Phe
        370 375
```

The deduced polypeptide of the shrimp actin consists of a 64-amino acid signal peptide and a 311-amino acid mature polypeptide. This shrimp actin exhibits 94% amino acid homology with the tiger prawn (Penaeus monodon) actin, 93% homology with the rattail fish (Coryphaenoides acrolepis) skeletal alpha actin type 2, and 93% homology with human (Homo sapiens) alpha actin of the cardiac muscle.

Also encompassed by the present invention are fragments and variants of the above nucleic acid molecules and the proteins or polypeptides they encode. Fragments of a nucleic acid molecule of the present invention may be made, for example, synthetically, or by use of restriction enzyme digestion on an isolated nucleic acid molecule. Variants may be made by the deletion or addition of amino acids that have minimal influence on the properties, secondary structure and hydropathic nature of the polypeptide. For example, a polypeptide may be conjugated to a signal (or leader) sequence at the N-terminal end of the protein which co-translationally or post-translationally directs transfer of the protein. The polypeptide may also be conjugated to a linker or other sequence for ease of synthesis, purification, or identification of the polypeptide.

Another aspect of the present invention relates to a nucleic acid construct containing the shrimp nucleic acid promoters of the present invention. This involves incorporating a nucleic acid promoter molecule of the present invention into host cells using conventional recombinant DNA technology. Generally, this involves inserting the nucleic acid molecule into an expression vector to which the nucleic acid molecule is heterologous (i.e., not normally present). A vector is generally constructed to include a promoter, a nucleic acid molecule targeted for transcription and/or expression, and a 3′ regulatory region having suitable transcriptional termination signals.

“Vector” is used herein to mean any genetic element, such as a plasmid, phage, transposon, cosmid, chromosome, virus, virion, etc., which is capable of replication when associated with the proper control elements, and which is capable of transferring gene sequences between cells. Thus, the term includes cloning and expression vectors, as well as viral vectors, including adenoviral and retroviral vectors.

Exemplary vectors include, without limitation, the following: lambda vector system gt11, gt WES.tB, Charon 4, and plasmid vectors such as pBR322, pBR325, pACYC177, pACYC184, pUC8, pUC9, pUC18, pUC19, pLG339, pR290, pKC37, pKC101, SV 40, pBluescript II SK+/− or KS+/− (see “Stratagene Cloning Systems” Catalog (1993) from Stratagene, La Jolla, Calif., which is hereby incorporated by reference in its entirety), pQE, pIH821, pGEX pET series (see F. W. Studier et. al., “Use of T7 RNA Polymerase to Direct Expression of Cloned Genes,” Gene Expression Technology Vol. 185 (1990), which is hereby incorporated by reference in its entirety), and any derivatives thereof. Recombinant genes may also be introduced into viruses, such as vaccinia virus. Recombinant viruses can be generated by transfection of plasmids into cells infected with virus.

Transcription of a target nucleic acid molecule in such a construct is dependent upon the presence of a promoter, which is a DNA sequence that directs the binding of RNA polymerase and thereby promotes mRNA synthesis. In this aspect of the present invention the promoter is the β-actin nucleic acid promoter molecule of the present invention having SEQ ID NO: 1. The β-actin and actin promoters of the present invention are a constitutive, non-inducible, non-developmental promoters. A constitutive promoter is a promoter that directs expression of a gene throughout the development and life of an organism. The promoters of the present invention are suitable, therefore, linked in the nucleic acid construct of the present invention to one or more nucleic acid molecules encoding a target protein or polypeptide of interest for which constitutive expression in the selected host is desired.

Any target nucleic acid molecule(s) of interest may be operably linked to this promoter molecule in a suitable vector, such that the nucleic acid molecule is under the control of the promoter of the present invention, including but not limited to, nucleic acids encoding viral proteins, such as coat proteins; growth regulating proteins, and proteins relating to enhanced stress tolerance in hosts transformed with such nucleic acid molecules, including heat shock proteins for increasing tolerance to cold-related stress.

Also present in the vector is a 3′ regulatory region containing suitable transcription termination signals selected from among those which are capable of providing correct transcription termination and polyadenylation of mRNA for expression in the host cell of choice, operably linked to a nucleic acid molecule which encodes for a protein or polypeptide of choice. Exemplary 3′ regulatory regions for the nucleic acid constructs of the present invention include, without limitation, the nopaline synthase (“nos”) 3′ regulatory region (Fraley, et al., “Expression of Bacterial Genes in Plant Cells,” Proc. Nat'l Acad. Sci. USA 80(15):4803-4807 (1983), which is hereby incorporated by reference in its entirety) and the cauliflower mosaic virus (“CaMV”) 3′ regulatory region (Odell, et al., “Identification of DNA Sequences Required for Activity of the Cauliflower Mosaic Virus 35S Promoter,” Nature 313(6005):810-812 (1985), which is hereby incorporated by reference in its entirety). An example of a commonly-used 3′ regulatory element for expression of genes of interest in animal cells is the SV40 polyadenylation signal derived from the SV40 virus. Virtually any 3′ regulatory element known to be operable in the host cell of choice will suffice for proper expression of the genes contained in the plasmids of the present invention.

Also suitable in the nucleic acid construct of the present invention is a reporter gene (marker gene) such as β-galactosidase, luciferase, or green fluorescent protein (GFP) or enhanced green fluorescent protein (EGFP) gene of the bioluminescent jelly fish, Aequorea victoria (Inoue, “Expression of Reporter Genes Introduced by Microinjection and Electroporation in Fish Embryos and Fry,” Mol. Mar. Biol. and Biotechnol. 1(4/5): 266-270 (1992); Boulo et al., “Transient Expression of Luciferase Reporter Gene After Lipofection in Oyster (Crassostrea gigas) Primary Cell Cultures,” Mol. Mar. Biol. Biotechnol. 5(3):167-74 (1996); Guillen et al., “Reporter Genes for Transgenic Fish Experiments,” Biotechnol. Apl. 13(4):279-283 (1996); Arnone et al., “Green Fluorescent Protein in the Sea Urchin: New Experimental Approaches to Transcriptional Regulatory Analysis in Embryos and Larvae,” Development 124(22):4649-4659 (1997); Husebye et al., “A Functional Study of the Salmon GnRH Promoter,” Mol. Mar. Biol. Biotechnol. 6(4):357-363 (1997); Joore et al., “Regulation of the Zebrafish Goosecoid Promoter by Mesoderm Inducing Factors and Xwnt1,” Mech. Dev. 55:3-18 (1997), which are hereby incorporated by reference in their entirety). A reporter gene is added to the nucleic acid construct of the present invention in order to evaluate the promoter's capacity to effectively direct expression of the target nucleic acid. Expression of the reporter gene is a good indication of whether the target gene was properly introduced into the host organism. The expression of the reporter gene also serves as a marker, helping to identify the organs and tissues in which the promoter is capable of driving target nucleic acid expression (Watson et al., “New Tools for Studying Gene Function,” In: Recombinant DNA, New York: Scientific American Books, pp. 191-272 (1992); Winkler et al., “Analysis of Heterologous and Homologous Promoters and Enhancers in vitro and in vivo by Gene Transfer Into Japanese Medaka (Oryzias latipes) and xiphophorus,” Mol. Mar. Biol. and Biotechnol. 1 (4/5):326-337 (1992), which are hereby incorporated by reference in their entirety). Expression of the β-galactosidase gene can be monitored easily via spectrophotometry and expression of the EGFP gene can be visualized directly in live, transparent, transgenic shrimp under a fluorescence microscope (Amsterdam et al., “The Aequorea Victoria Green Fluorescent Protein Can be Used as a Reporter in Live Zebrafish Embryos,” Dev. Biol. 171(1):123-129 (1995); Kain et al., “Green Fluorescent Protein as a Reporter of Gene Expression and Protein Localization,” Biotechniques 19(4):650-655 (1995); Burlage et al., “Green Fluorescent Protein in the Sea Urchin: New Experimental Approaches to Transcriptional Regulatory Analysis in Embryos and Larvae,” Development 124(22):4649-4659 (1997); Hong et al., “Dynamics of Nontypical Apoptotic Morphological Changes Visualized by Green Fluorescent Protein in Living Cells with Infectious Pancreatic Necrosis Virus Infection,” J. Virol. 73(6):5056-63 (1999), which are hereby incorporated by reference in their entirety) or hand-held UV Lamp (Clare Chemical Research).

The promoter molecule of the present invention, a nucleic acid molecule encoding a protein or polypeptide of choice, a suitable 3′ regulatory region, and if desired, a reporter gene, are incorporated into a vector-expression system of choice to prepare the nucleic acid construct of present invention using standard cloning procedures known in the art, such as described by Sambrook et al., Molecular Cloning: A Laboratory Manual, Third Edition, Cold Spring Harbor: Cold Spring Harbor Laboratory Press, New York (2001), which is hereby incorporated by reference in its entirety, and U.S. Pat. No. 4,237,224 to Cohen and Boyer, which is hereby incorporated by reference in its entirety, which describes the production of expression systems in the form of recombinant plasmids using restriction enzyme cleavage and ligation with DNA ligase. These recombinant plasmids are then introduced by means of transformation and replicated in unicellular cultures including prokaryotic organisms and eukaryotic cells grown in tissue culture.

In one aspect of the present invention, a nucleic acid molecule encoding a protein of choice is inserted into a vector in the sense (i.e., 5′→3′) direction, such that the open reading frame is properly oriented for the expression of the encoded protein under the control of a promoter of choice. Single or multiple nucleic acids may be ligated into an appropriate vector in this way, under the control of one of the promoters of the present invention.

In another aspect of the present invention, a target nucleic acid encoding a protein of choice is inserted into the vector in an antisense orientation (3′→5′). The use of antisense RNA to down-regulate the expression of specific plant genes is well known (van der Krol et al., “Antisense Genes in Plants: An Overview,” Gene 72:45-50 (1988); van der Krol et al., “Inhibition of Flower Pigmentation by Antisense CHS Genes: Promoter and Minimal Sequence Requirements for the Antisense Effect,” Plant Mol Biol 14(4):457-66 (1990); Mol et al., “Regulation of Plant Gene Expression by Antisense RNA,” FEBS Lett 286:427-430 (1990); and Smith et al., Nature, 334:724-726 (1988); which are hereby incorporated by reference in their entirety). Antisense nucleic acids are DNA or RNA molecules that are complementary to at least a portion of a specific mRNA molecule (Weintraub, “Antisense RNA and DNA,” Scientific American 262:40 (1990), which is hereby incorporated by reference in its entirety). Antisense methodology takes advantage of the fact that nucleic acids tend to pair with “complementary” sequences. By complementary, it is meant that polynucleotides are capable of base-pairing according to the standard Watson-Crick rules. In the target cell, the antisense nucleic acids hybridize to a target nucleic acid and interfere with transcription, and/or RNA processing, transport, translation, and/or stability. The overall effect of such interference with the target nucleic acid function is the disruption of protein expression.

Accordingly, both antisense and sense forms of the nucleic acids of the present invention are suitable for use in the nucleic acid constructs of the invention. A single construct may contain both sense and antisense forms of one or more desired nucleic acids encoding a protein.

Alternatively, the nucleic acid construct of the present invention may be configured so that the DNA molecule encodes an mRNA which is not translatable, i.e., does not result in the production of a protein or polypeptide. This is achieved, for example, by introducing into the desired nucleic acid sequence of the present invention one or more premature stop codons, adding one or more bases (except multiples of 3 bases) to displace the reading frame, and removing the translation initiation codon (U.S. Pat. No. 5,583,021 to Dougherty et al., which is hereby incorporated by reference in its entirety). This can involve the use of a primer to which a stop codon, such as TAA or TGA, is inserted into the sense (or “forward”) PCR-primer for amplification of the fill nucleic acid, between the 5′ end of that primer, which corresponds to the appropriate restriction enzyme site of the vector into which the nucleic acid is to be inserted, and the 3′ end of the primer, which corresponds to the 5′ sequence of the enzyme-encoding nucleic acid.

Genes can be effective as silencers in the non-translatable antisense forms, as well as in the non-translatable sense form (Baulcombe, D. C., “Mechanisms of Pathogen-Derived Resistance to Viruses in Transgenic Plants,” Plant Cell 8:1833-44 (1996); Dougherty et al., “Transgenes and Gene Suppression: Telling us Something New?” Current Opinion in Cell Biology 7:399-05 (1995); Lomonossoff, G. P., “Pathogen-Derived Resistance to Plant Viruses,” Ann. Rev. Phytopathol. 33:323-43 (1995), which are hereby incorporated by reference in their entirety). Accordingly, one aspect of the present invention involves nucleic acid constructs which contain one or more of the nucleic acid molecules of the present invention as a nucleic acid which encodes a non-translatable mRNA, that nucleic acid molecule being inserted into the construct in either the sense or antisense orientation. Several vectors have been constructed using the β-actin nucleic acid promoter of the present invention, as detailed below in the Examples.

Once the nucleic acid construct of the present invention has been prepared, it is ready to be incorporated into a host cell. Accordingly, another aspect of the present invention relates to a recombinant cell, or “hos” cell containing a nucleic acid construct of the present invention. A variety of vector-host systems known in the art may be utilized to express the protein-encoding sequence(s). Primarily, the vector system must be compatible with the host cell used. Host-vector systems include, but are not limited to, the following: bacteria transformed with bacteriophage DNA, plasmid DNA, or cosmid DNA; microorganisms such as yeast containing yeast vectors; mammalian cell systems infected with virus (e.g., vaccinia virus, adenovirus, etc.); insect cell systems infected with virus (e.g., baculovirus); and animal cells, including marine fish, crustacean, particularly shrimp, and other marine animals, infected by bacterial vector. Host cells are prepared by delivery of vector into the host organism.

Three common methods of vector-expression for foreign nucleic acid delivery are electroporation (Muller et al., “Introducing Foreign Genes Into Fish Eggs With Electroporated Sperm as a Carrier,” Mol. Mar. Biol. Biotechnol. 1:276-281 (1992); Powers et al., “Electroporation: a Method for Transferring Genes Into the Gametes of Zebra Fish (Brachydanio rerio), Channel Catfish (Ictalurus punctatus), and Common Carp (Cyprimus cario),” Mol. Mar. Biol. Biotechnol. 1:301-308 (1992); Sin et al., “Gene Transfer in Chinook Salmon by Electroporating Sperm in the Presence of PRSV-lacZ DNA,” Aquaculture 117:57-69 (1993); Powers et al., “Electroporation as an Effective Means of Introducing DNA Into Abalone (Haliotis rufescens) Embryos,” Mol. Mar. Biol. Biotechnol. 4(4):369-375 (1995); Tsai et al., “Sperm as a Carrier to Introduce an Exogenous DNA Fragment Into the Oocyte of Japanese Abalone (Haliotis divorsicolor suportexta),” Transgenic Res. 6(1):85-95 (1997); Fraga et al., “Introducing Antisense Oligonucleotides into Paramecium via Electroporation,” J. Eukaryot. Microbiol. 45(6):582-8 (1998); Preston et al., “Delivery of DNA to Early Embryos of the Kuruma Prawn, Penaeus japonicus,” Aquaculture 181:225-234 (2000), which are hereby incorporated by reference in their entirety), ballistic bombardment (Zelenin et al., “The Delivery of Foreign Genes Into Fertilized Eggs Using High-Velocity Microprojectiles,” FEBS Lett. 287(1-2):118-120 (1991); Akasaka et al., “Introduction of DNA Into Sea Urchin Eggs by Particle Gun,” Mol. Mar. Biol. Biotechnol. 4(3):255-261 (1995); Gendreau et al., “Transient Expression of a Luciferase Reporter Gene After Ballistic Introduction Into Artemia Franciscana (Crustacea) Embryos,” Aquaculture 133:199-205 (1995); Baum et al., “Improved Ballistic Transient Transformation Conditions for Tomato Fruit Allow Identification of Organ-Specific Contributions of I-Box and G-Box to the RBCS2 Promoter Activity,” Plant J. 12(2):463-9 (1997); Udvardi et al., “Uptake of Exogenous DNA Via the Skin,” J. Mol. Med. 77(10):744-50 (1999), which are hereby incorporated by reference in their entirety) and microinjection (Udvardi et al., “Uptake of Exogenous DNA Via the Skin,” J. Mol. Med. 77(10):744-50 (1999); Penman et al., “Patterns of Transgene Inheritance in Rainbow Trout (Oncorhynchus Mykiss),” Mol. Reprod. Dev. 30:201-206 (1991); Damen et al., “Transcriptional Regulation of Tubulin Gene Expression in Differentiating Trochoblasts During Early Development of Patella Vulgata,” Development 120:2835-2845 (1994); Gaiano et al., “Highly Efficient Germ-Line Transmission of Proviral Insertions,” Proc. Natl. Acad. Sci. USA 93:7777-7782 (1996); Cadoret et al., “Microinjection of Bivalve Eggs: Application in Genetics,” Mol. Mar. Biol. Biotechnol. 6(1):7277 (1997); Li et al., “Transfer of Foreign Gene to Giant Freshwater Prawn (Macrobrachium rosenbergii) by Spermatophore-Microinjection,” Mol. Rerod. Dev. 56(2):149-54 (2000), which are hereby incorporated by reference in their entirety). Among these three methods, microinjection is considered to be the most tedious, but most efficient, method for transferring foreign nucleic acid into marine and fresh water species. It allows precision in delivery of exogenous nucleic acid and increases the chances that a treated egg will be transformed. The introduced nucleic acid is ultimately integrated into the chromosomes of the microinjected organism. Preston et al., “Delivery of DNA to Early Embryos of the Kuruma Prawn, Penaeus japonicus,” Aquaculture 181:225-234 (2000) (which is hereby incorporated by reference in its entirety), examined the relative efficiency of microinjection, electroporation, and particle bombardment for introducing nucleic acid into the embryos of the Kuruma prawn, Litopenaeus japonicus and found that microinjection is the most reliable technique but very time consuming. Electroporation is a desirable method for large scale gene transfer, however, if the host mortality is high, an alternative non-surgical technique (e.g., spermatophore-microinjection), can be used as the delivery system. While stable expression is generally preferable, transient expression is suitable for some uses of the nucleic acid constructs of the present invention, therefore, the choice of delivery system in this aspect of the invention may vary depending on the type of expression desired.

After transformation, the transformed host cells can be selected and expanded in suitable culture. Preferably, transformed cells are first identified using a selection marker simultaneously introduced into the host cells along with the nucleic acid construct of the present invention. Suitable markers include those genes described above as reporter genes, i.e., β-glucuronidase, luciferase, EGFP, or additionally, markers encoding for antibiotic resistance, such as the nptII gene which confers kanamycin resistance (Fraley, et al., “Expression of Bacterial Genes in Plant Cells,” Proc. Nat'l Acad. Sci. USA 80(15):4803-4807 (1983), which is hereby incorporated by reference in its entirety), or gentamycin, G418, ampicillin, streptomycin, spectinomycin, tetracycline, chloramphenicol, and the like. A number of antibiotic-resistance markers are known in the art and others are continually being identified. Any known antibiotic-resistance marker can be used to transform and select transformed host cells in accordance with the present invention. Cells or tissues are grown on a selection medium containing an antibiotic, whereby generally only those transformants expressing the antibiotic resistance marker continue to grow. Similarly, enzymes providing for production of a compound identifiable by luminescence, such as luciferase, are useful. The selection marker employed will depend on the target species; for certain target species, different antibiotics, or biosynthesis selection markers are preferred.

The present invention also relates to a transgenic animal transformed with a nucleic acid construct of the present invention described above having a nucleic acid molecule encoding a protein under the control of the β-actin or actin promoter of the present invention. This involves preparing a nucleic acid construct as described above containing the β-actin or actin promoter, a nucleic acid molecule encoding a desired protein, and a 3′ regulatory region for termination, incorporating the nucleic acid construct into a suitable vector-host system, and transforming an animal using a suitable delivery system, such as those described above. Animals suitable for this aspect of the present invention include, without limitation, marine fish; crustaceans, including shrimp and prawns; shellfish; and insects.

When stable transformation of a transgenic animal is achieved, the gene is incorporated into the organism's genome, and the gene is, therefore, heritable. Accordingly, the present invention also relates to the progeny of the a transgenic animal transformed with the nucleic acid construct described above having a nucleic acid molecule encoding a protein under the control of the β-actin or actin promoter of the present invention, wherein the progeny harbors the transformed nucleic acid.

Another aspect of the present invention is a nucleic acid expression cassette including a β-actin promoter molecule isolated from shrimp having SEQ ID NO: 1; a multiple cloning site; an operable termination segment; and a nucleic acid molecule encoding a detectable marker. In this aspect, a nucleic acid expression cassette is prepared generally as described for the making of the nucleic acid construct having the β-actin promoter of the present invention, with the promoter molecule and a suitable 3′ termination segment (meaning a polyadenylation signal and a termination signal). However, the promoter is incorporated into a vector having a multiple cloning site (MCS) for the insertion of one or more nucleic acid molecules of choice by a user. In one embodiment the expression cassette also contains a detectable marker. Exemplary markers include, without limitation, those named above. The promoter molecule, a suitable 3′ termination segment, and, if desired, a detectable marker, are ligated into a vector having a MCS, using standard cloning procedures known in the art, such as described by Sambrook et al., Molecular Cloning: A Laboratory Manual, Third Edition, Cold Spring Harbor: Cold Spring Harbor Laboratory Press, New York (2001), and U.S. Pat. No. 4,237,224 to Cohen and Boyer, which are hereby incorporated by reference in their entirety.

The present invention also relates to a method of imparting to an animal resistance against a pathogen. This involves transforming an animal with the nucleic acid construct of the present invention described above having the actin or β-actin promoter of the present invention, a nucleic acid molecule encoding a protein for resistance to a pathogen, and an operable 3′ regulatory region. In one aspect of the present invention, the pathogen is a virus. Exemplary viruses against which resistance is imparted include those selected from the group consisting of white spot syndrome virus (WSSV), yellow head virus (YHV), Taura syndrome virus (TSV), and infectious hypodermal and hematopoietic necrosis virus (IHHNV). In one embodiment of the present invention, the nucleic acid molecule encodes a viral coat protein, or a fragment thereof. Suitable nucleic acid molecules are those encoding for the viral coat protein or polypeptide of (WSSV), (YHV), (TSV), and (IHHNV). One or more coat protein-encoding nucleic acid molecules can be used in a single construct, so as to confer resistance to multiple viruses to one animal with a single vector.

While not wishing to be bound by theory, by use of the constructs of the present invention, it is believed that viral resistance transgenic animals can result using RNA-mediated post-transcriptional gene silencing. The strategy is to introduce a transgene consisting of sense and/or antisense versions of target gene (for examples, TSV coat protein and the IHHNV coat protein) fragments into a host animal, so that the expressed RNA transcripts will interfere with the translation process of the TSV and IHV coat protein genes, thereby inhibiting viral replication in the animal.

More particularly, the silencer DNA molecule is believed to boost the level of heterologous RNA within the cell above a threshold level. This activates the degradation mechanism by which viral resistance is achieved.

Posttranscriptional gene silencing (PTGS) based on RNA interference (RNAi) destroys RNA in a sequence-specific manner (Baulcombe, “RNA Silencing,” Curr. Biol. 12(3):R82-4 (2002); Hutvagner et al., “RNAi: Nature Abhors a Double-Strand,” Curr. Opin. Genet. Dev. 12(2):225-232 (2002), Hutvagner et al., “A MicroRNA in a Multiple-Turnover RNAi Enzyme Complex,” Science 297(5589):2056-2060 (2002), which are hereby incorporated by reference in their entirety) and functions in the natural immunity of animal cells. Significant progress in the area of viral resistance through RNA-mediated gene silencing has been achieved through research of RNAi in plants (Waterhouse et al., “Virus Resistance and Gene Silencing in Plants Can be Induced by Simultaneous Expression of Sense and Antisense RNA,” Proc. Natl. Acad. Sci. USA 95(23):13959-64 (1998); Pang et al., “Resistance to Squash Mosaic Comovirus in Transgenic Squash Plants Expressing its Coat Protein Genes,” Mol. Breed. 6:87-93 (2000); Vance et al., “RNA Silencing in Plants—Defense and Counterdefense,” Science 292(5525):2277-2280 (2001); Hongwei et al., “Induction and Suppression of RNA Silencing by Animal Virus,” Science 296:1319-1321 (2002), which are hereby incorporated by reference in their entirety) and animals (Takayama et al., “Antisense RNA-Mediated Inhibition of Viral Infection in Tissue Culture and Transgenic Mice,” In: Molecular Biology of RNA, Less, (ed.), pp. 299-310. New York (1989); Kim et al., “Examination of Antisense RNA and Oligodeoxynucleotides as Potential Inhibitors of Avian Leukosis Virus Replication in RP30 Cells,” Poultry Sci. 77:1400-10 (1998); Player et al., “Potent Inhibition of Respiratory Syncytial Virus Replication Using a 2-5A-Antisense Chimera targeted to Signals Within the Virus Genomic RNA,” Proc. Natl. Acad. Sci. USA 95:8874-9 (1998); Knight et al., “A Role for the RNase III Enzyme DCR-1 in RNA Interference and Germ Line Development in Caenorhabditis Elegans,” Science 293(5538):2269-2271 (2001); Tang et al., “Detection and Quantification of Infectious Hypodermal and Hematopoietic Necrosis Virus in Penaeid Shrimp by Real-Time PCR,” Dis. Aquat. Org. 44(2):79-85 (2001); Korneev et al., “Suppression of Nitric Oxide (NO)-Dependent Behavior by Double-Stranded RNA-Mediated Silencing of a Neuronal NO Synthase Gene,” J. Neurosci. 22(11):RC227 (2002); Gitlin et al., “Short Interfering RNA Confers Intracellular Antiviral Immunity in Human Cells,” Nature, Online publication (2002), which are hereby incorporated by reference in their entirety). Current review of RNA-mediated gene silencing mechanisms have been extensively described (Ahlquist, “RNA-Dependent RNA Polymerases, Viruses, and RNA Silencing,” Science 296:1270-1273 (2002); Plasterk, 2002, which are hereby incorporated by reference in their entirety). Examples of transgenic animals include: inhibition of Moloney murine leukemia virus in mice with anti-sense RNA against the retroviral packaging sequences (Han et al., “Inhibition of Moloney Murine Leukemia Virus-Induced Leukemia in Transgenic Mice Expressing Antisense RNA Complementary to the Retroviral Packaging Sequences,” Proc. Natl. Acad. Sci. USA 88:4313-17 (1991), which is hereby incorporated by reference in its entirety), transgenic mice resistant to hepatitis virus (Sasaki et al., “Transgenic Mice With Antisense RNA Against the Nucleocapsid Protein mRNA of Mouse Hepatitis Virus,” J. Vet. Med. Sci. 55(4):549-54 (1993), which is hereby incorporated by reference in its entirety), and Aedes aegypti mosquitoes resistant to luciferase expression (Johnson et al., “Inhibition of Luciferase Expression in Transgenic Aedes Aegypti Mosquitoes by Sindbis Virus Expression of Antisense Luciferase RNA,” Proc. Natl. Acad. Sci. USA 96(23): 13399-403 (1999), which is hereby incorporated by reference in its entirety). Generally, pathogen resistance was mediated through production of viral coat protein RNA in the above listed studies. Viral coat protein genes, and fragments thereof, have been used successfully in plants for RNA-mediated pathogen-derived resistance since presumably, the transcript is highly expressed and is very stable (Pang et al., “Nontarget DNA Sequences Reduce the Transgene Length Necessary for RNA-Mediated Tospovirus Resistance in Transgenic Plants,” Proc. Natl. Acad. Sci. USA 94:8261-8266 (1997), which is hereby incorporated by reference in its entirety). It was demonstrated that only a portion of the coat protein gene was required to confer resistance against the viral pathogen. For example, a minimum length (somewhere between 236-387 bp) of the gene for the 29 Kd nucleocapsid protein of tomato spotted wilt virus (TSWV) was required to develop RNA-mediated resistance in transgenic Nicotiana benthamiana plants (Pang et al., “Nontarget DNA Sequences Reduce the Transgene Length Necessary for RNA-Mediated Tospovirus Resistance in Transgenic Plants,” Proc. Natl. Acad. Sci. USA 94:8261-8266 (1997), which is hereby incorporated by reference in its entirety). It was also determined that any region of the coding sequence for the TSWV nucleocapsid protein can be used to develop virus resistance (Pang et al., “Nontarget DNA Sequences Reduce the Transgene Length Necessary for RNA-Mediated Tospovirus Resistance in Transgenic Plants,” Proc. Natl. Acad. Sci. USA 94:8261-8266 (1997), which is hereby incorporated by reference in its entirety).

Animals suitable for this aspect of the present invention include, without limitation, those selected from the group consisting of marine fish; crustaceans, including prawns and shrimp; shellfish; and insects.

The present invention also relates to a method of regulating the growth of an animal. This involves transforming an animal with a nucleic acid construct of the present invention having the actin or β-actin promoter of the present invention operably linked to a nucleic acid molecule encoding a growth regulating protein, and a 3′ regulatory region. Nucleic acid molecules suitable for this aspect of the present invention include those that encode proteins that up-regulate growth and down-regulate growth. Examples of suitable proteins that can be used to up-regulate growth include growth hormones, including without limitation, the androgenic hormone. Animals suitable for this aspect of the present invention include, without limitation, those selected from the group consisting of marine fish; crustaceans, including prawns and shrimp; shellfish; and insects.

Another aspect of the present invention is a method of increasing stress tolerance in an animal, including stress induced by cold. This involves transforming an animal with the nucleic acid construct of the present invention having the actin or β-actin promoter of the present invention operably linked to a nucleic acid molecule encoding protein and a 3′ regulatory region. Nucleic acid molecules suitable for this aspect of the present invention include those encoding for a protein that increases stress tolerance in an animal. An exemplary protein would be a heat shock protein, such as HSP70 or HSP26, which may enhance cold tolerance in an animal. Animals suitable for this aspect of the present invention include without limitation, those selected from the group consisting of marine fish; crustaceans, including prawns and shrimp; shellfish; and insects.

The present invention also relates to a nucleic acid construct having the isolated nucleic acid molecule encoding β-actin from shrimp having a nucleotide sequence of SEQ ID NO: 2, and an expression vector and host cells transduced with such a nucleic acid construct. In this aspect of the present invention, preparation of nucleic acid construct, vector, and host cells is carried out as described above for nucleic acid constructs, vector, and host cells in earlier aspects of the present invention, including the choice of suitable vectors, 3′ regulatory regions, other regulatory element(s) when appropriate, and host cells, or in accordance with molecular biology methods available in the art, with the exception of the nucleic acid promoter molecule. The nucleic acid promoter molecule used in the nucleic acid construct of this aspect of the present invention may be one of the promoter molecules of the present invention, for example, the actin or β-actin nucleic acid promoters of the present invention. Other promoters are also suitable, including those that are constitutive, inducible or repressible. Examples of some constitutive promoters that are widely used for inducing expression of transgenes include the nopoline synthase (“NOS”) gene promoter, from Agrobacterium tumefaciens, (U.S. Pat. No. 5,034,322 to Rogers et al., which is hereby incorporated by reference in its entirety), the cauliflower mosaic virus (“CaMV”) 35S and 19S promoters (U.S. Pat. No. 5,352,605 to Fraley et al., which is hereby incorporated by reference in its entirety), the enhanced CaMV35S promoter (“enh CaMV35S”). Also suitable are those derived from any of the several previously identified actin genes, which are known to be expressed in most cells types (U.S. Pat. No. 6,002,068 to Privalle et al., which is hereby incorporated by reference in its entirety), and the ubiquitin promoter (“ubi”), which is a gene product known to accumulate in many cell types. Promoters for this aspect of the present invention are chosen with regard to the desired application of the nucleic acid construct, and are incorporated into the nucleic acid construct as described above or by using standard cloning procedures known in the art, such as described by Sambrook et al., Molecular Cloning: A Laboratory Manual, Third Edition, Cold Spring Harbor: Cold Spring Harbor Laboratory Press, New York (2001), and U.S. Pat. No. 4,237,224 to Cohen and Boyer, which are hereby incorporated by reference in their entirety.

Another aspect of the present invention relates to a nucleic acid expression cassette containing an isolated actin nucleic acid promoter molecule of the present invention, a multiple cloning site, an operable termination segment, and a nucleic acid molecule encoding a detectable marker. In this aspect, a nucleic acid expression cassette is prepared generally as described for the making of the nucleic acid construct having the actin promoter of the present invention, with the promoter molecule and a suitable 3′ termination segment (meaning a polyadenylation signal and a termination signal); however, the promoter is incorporated into a vector having a multiple cloning site (MCS) for the insertion of one or more nucleic acid molecules of choice by a user. In one embodiment, the expression cassette also contains a detectable marker. Exemplary markers include, without limitation, green fluorescent protein, enhanced green fluorescent protein, β-galactosidase, and luciferase. The promoter molecule, 3′ termination segment, and detectable marker, if desired, are ligated into a vector having a MCS, using standard cloning procedures known in the art, such as described by Sambrook et al., Molecular Cloning: A Laboratory Manual, Third Edition, Cold Spring Harbor: Cold Spring Harbor Laboratory Press, New York (2001), and U.S. Pat. No. 4,237,224 to Cohen and Boyer, which are hereby incorporated by reference in their entirety.

### EXAMPLES

### Example 1

### Animals

Live shrimp and fertilized eggs of L. vannamei were obtained from a local aquafarm in Hawaii. Immediately after fertilization, the shrimp eggs were collected with a fine mesh net and concentrated by a brief centrifugation at 1000 g for 20 seconds, then transferred to 1.5 ml sterilized sea water in a small dish and subjected to micro-injection.

### Example 2

### Micro-Injection Procedures

Procedures of micro-injection followed the methods of Chong et al., “Expression and Fate of CAT Reporter Gene Microinjected into Fertilized Medaka (Oryzias latipes) Eggs in the Form of Plasmid DNA, Recombinant Phage Particles and its DNA,” Theor. Appl. Genet. 78:369-380 (1989), Penman et al., “Factors Effecting Survival and Integration Following Micro-Injection of Novel DNA in Rainbow Trout Eggs,” Aquaculture 85:35-50 (1990); Collas et al., “Transferring Foreign Genes into Zebrafish Eggs By Microinjection,” In Houdebine, L. M. (ed.) Transgenic Animals—Generation and Use Harwood Academic Publishers (In Press); which are hereby incorporated by reference in their entirety), with modifications. Briefly, microinjection was performed with the Femtojet microinjection system (Brinkmann Instruments, Inc., Westbury, N.Y.). Femtotip injection needles (Brinkmann Instruments, Inc.) were secured to the micromanipulator (Drummond Scientific Co., Philadelphia, Pa.) made from borosilicate glass capillary tubes using a horizontal Sutter P-87 puller. The needles were generally about 5 cm long, steep near the shoulder, and shallow close to the tip which is of 10-15 μm in diameter. Mineral oil was introduced into the base of the injection needle prior to being secured to the micro-manipulator (Drummond Scientific Co., Philadelphia, Pa.). DNA solution (1 μg/μl in sterilized H₂O) was introduced into the needle with a microloader tip (Brinkmann Instruments, Inc.) through the tip by capillary action. Prior to injection, the shrimp eggs were placed in the petri dish equipped with a fine mesh grid, which serves as a barrier so that the eggs will not roll during injection. The petri dish was then placed under a high power stereo dissecting microscope (Carl Zeiss, Jena, Germany) with the injection needle positioned at 45° above the horizontal axis. Eggs were injected with approximately 50 picoliters of DNA solution. Each injector dispenser released 4 ηl (working range is 4 ηl -40 η1) of DNA solution into the egg. With suitable adjustment of the micro-manipulator, a suitable rate of injection of about 5 injections per minute was achieved. Several hundred eggs can be injected per hour with a single needle filling. A series of micro-injection experiments were performed for testing transgene expression efficiency of several constructs which contain various regulatory regions of the shrimp β-actin5C gene.

### Example 3

### Sample Collection

Immediately following injection, the putative transformed eggs were placed in a one-liter container with aerated seawater at room temperature where hatching takes place in about one day. After hatching, larvae were transferred to a 5-gallon glass aquarium (16″L×8″W×10″H) with aerated seawater containing 0.15 ppm each of penicillin and streptomycin. Control groups of shrimp eggs were treated identically except for injection with water alone. The techniques for raising penaeid shrimp from the egg to post-larvae generally followed the methods described by Mock et al., “Techniques for Raising Penaeid Shrimp from the Egg to Postlarvae,” Maricult. Proc. World Soc. 3:143-156 (1972), Brown et al., “The Maturation and Spawning of Penaeus stylirostris Under Controlled Laboratory Conditions,” Proc. World Maricult. Soc. 11:488-499 (1980), and Wyban et al., “Intensive Shrimp Growth Trials in a Round Pond,” Aquaculture 76:215-225 (1989) (which are hereby incorporated by reference in their entirety). The shrimp were fed with algae feed, and observed closely throughout the experimental period. Animals were examined or sacrificed at several stages during growth and development for analysis of transgene expression.

### Example 4

### Reverse Transcription-Polymerase Chain Reaction

Live shrimp (L. vannamei) were obtained from a local aquafarm in Hawaii. Approximately 2 μg of total RNA from shrimp tissue was used for reverse transcription reaction, and RT-PCR assays were carried out according to the protocol of the GeneAmp RNA PCR Kit (Perkin-Elmer Cetus, Norwalk, Conn.), with slight modifications as described previously (Sun, “Molecular Cloning and Sequence Analysis of a cDNA Encoding a Molt-Inhibiting Hormone-like Neuropeptide from the White Shrimp Penaeus vannamei,” Mol. Mar. Biol. Biotechnol. 3(l):1-6 (1994), which is hereby incorporated by reference in its entirety). Amplifications were performed by a DNA Thermal Cycler (Perkin-Elmer 9600) programmed at suitable temperatures for annealing and extension. A pair of degenerate primers (P1 and P2) were constructed based on unique sequences to cytoplasmic actin5C protein of D. melanogaster (Fyrberg et al., “The Actin Genes of Drosophila: Protein Coding Regions are Highly Conserved but Intron Positions Are Not,” Cell 24: 107-116 (1981); Bond et al., “The Drosophila Melanogaster Actin 5C Gene Uses Two Transcription Initiation Sites and Three Polyadenylation Sites to Express Multiple mRNA Species,” Mol. Cell Biol. 6(6):2080-2088 (1986), which are hereby incorporated by reference in their entirety) and were used for PCR amplification. The oligonucleotide sequences of P1 (1735-1762) and P2 (1959-1933) are as follows:

(SEQ ID NO: 7) sense;

| | |
|---|---|
| P1: | 5′CTTACAAAATGTGT(C)GAC(T)GAA(G)GAA(G)GTIGC 3′ |

(SEQ ID NO: 8) antisense

| | |
|---|---|
| P2: | 5′CCG(A)TGC(T)TCG(AT)ATIGGG(A)TAC(T)TTIAGIGT 3′ |

### Example 5

### Southern Hybridization

The PCR-amplified DNA products (10 μl) were separated by electrophoresis in a 2% low melting temperature agarose gel containing ethidium bromide (0.5 μg/ml). After electrophoresis, the DNA was transferred to Hybond-N⁺ membrane (Amersham, Piscataway, N.J.). Hybridization was performed at 42° C. for 18 hours with α³²P-labeled actin5C-cDNA probe (actin5C-cDNA from Drosophila) in a solution containing 50% formamide, 6×SSPE (1×SSPE=0.15 MNaCl, 10 mMNaH₂PO₄, 1 mM ethylenediaminetetraacetic acid, pH 7.4), 5× Denhardt's reagent, 0.5% sodium dodecyl sulfate (SDS), and 100 μg/100 ml denatured salmon sperm DNA with an actin5C-cDNA labeled by random priming with α³²P-dCTP as probe. After hybridization, the filter was washed two times for 15 minutes in 2×S SSPE and 0.2% SDS at 42° C., then two times for 15 minutes in 0.1×SSPE and 0.1% SDS at 68° C., and exposed to Kodak XAR-5 film at −80° C. for 10 hours.

### Example 6

### Cloning and Sequencing of DNA

The target DNA fragment, as identified by Southern hybridization, was cloned with the TA cloning kit (Invitrogen, Carlsbad, Calif.). Briefly, the PCR-product was ligated into the TA cloning vector, pCRII. One Shot competent cells were used for transformation. Positive white colonies were picked and analyzed by miniprep to verify the presence of cloned PCR product. Standard protocols for ligation, cloning, and transformation followed Sambrook et al., Molecular Cloning, A Laboratory Manual, Second edition, New York: Cold Spring Harbor Laboratory Press (1989). After purification using a QIAprep Spin Plasmid Miniprep Kit (Qiagen, Valencia, Calif.), DNA was sequenced by the dideoxy-chain-termination method (Sanger et al., “DNA Sequencing with Chain-Terminating Inhibitors,” Proc. Natl. Acad. Sci. USA 74:5463-5467 (1977), which is hereby incorporated by reference in its entirety) using the Sequenase system (version 2.0, USB, West Conshocken, Pa.) and/or the automatic sequencing method using the DyeDeoxy Terminator cycle sequencing kit (Applied Biosystems, Foster City, Calif., model 373A). Each DNA sample was sequenced twice in each direction for sequence confirmation.

### Example 7

### Library Screening

The existing shrimp genomic library, constructed using the LambdaGEM-11 vector and containing 360,000 recombinant clones, was first used for screening the genomic clone of actin5C. The relevant facts are that the actin5C gene is abundant in cytoplasm. Therefore, it was probable that the gene was present in the partial genomic library of 360,000 recombinant clones, and the vector is LambdaGEM-11 which contains the lengths of inserts between 9-23 kb. The known actin5C of Drosophila is 17.5 kb.

The genomic library was screened using a combination of PCR amplification (Amaravadi et al., “A Rapid and Efficient, Nonradioactive Method for Screening Recombinant DNA Libraries,” Biotechniques 16(l):98-103 (1994), which is hereby incorporated by reference in its entirety) and the in situ plaque hybridization technique (Benton et al., “Screening Lambda gt Recombinant Clones by Hybridization to Single Plaques in situ,” Science 196(4286):180-182 (1977), which is hereby incorporated by reference in its entirety) using primer 1 and primer 2 (described in Example 4) and the RT-PCR generated DNA fragment as a probe. Briefly, about 1×10⁶ recombinant clones were plated on 20 (150 mm) plates and incubated for 7-10 hours at 37° C. or until plaques begin to contact each other. The phages were soaked in 10 ml of phage diluted buffer (PDB) overnight at 4° C., the PDB collected from each plate, and centrifuged at 5,000×g for 10 min to remove debris. E. coli were lysed by adding a few drops of CHCl₃. An aliquot (1 μl) of plate lysate was used as the template for PCR assay. The PCR protocol was performed as previously described (Sun, “Molecular Cloning and Sequence Analysis of a cDNA Encoding a Molt-Inhibiting Hormone-like Neuropeptide from the White Shrimp Penaeus vannamei,” Mol. Mar. Biol. Biotechnol. 3(1):1-6 (1994), which is hereby incorporated by reference in its entirety) and the PCR products were first analyzed by agarose gel electrophoresis. The detection of an expected 224-bp DNA product indicated a positive actin5C clone in the plate lysate. Once a positive plate lysate was identified, several rounds of replating and PCR amplification led to the identification of individual positive plaques. Individual positive plaques were confirmed by plaque hybridization as described by Sambrook et al., Molecular Cloning. A Laboratory Manual, 2^{nd} ed., Cold Spring Harbor, N.Y.: Cold Spring Harbor Laboratory Press (1989), which is hereby incorporated by reference in its entirety, using the PCR-generated DNA labeled with α³²P-dCTP as a probe. The sensitivity and reliability of plaque hybridization demonstrated that a positive actin5C clone was obtained.

Phagemid DNA from positive clones were isolated using Wizard Lamb & Preps DNA Purification system (Promega, Madison, Wis.), and subjected to Southern analysis. Positive clones having the largest size of DNA were selected and their DNAs multiplied in E. coli, purified, and the DNAs were further characterized by sequencing and physical mapping. The nucleotide sequence of the shrimp actin5C gene was also analyzed for transcription factor binding motifs using the Find patterns and for sequence comparison with the chicken β-actin promoter and fly β-actin promoters using the Best-Fit program (Genetics Computer Group, Madison, Wis.).

For information purposes, the shrimp cDNA library was also screened for cDNA(s) encoding the actin5C protein using the same probe and strategies. The shrimp actin5C-cDNA(s) isolated from positive clones was purified and sequenced and their deduced amino acid sequences analyzed and compared with published data from other species.

### Example 8

### Mapping of the Transcriptional Start Site by Primer Extension

To determine the transcription start site, primer extension was performed using a AMV-reverse transcriptase primer extension system (Promega, Madison, Wis.). A 5′-end-labeled antisense oligonucleotide complementary to the part of the 5′-flanking region of the shrimp actin5C gene was incubated with 30 μg of total RNA isolated from shrimp embryos for 24 hours. After annealing at 62° C. for 20 minutes, AMV-reverse transcriptase extension mix was added to the annealed primer/RNA followed by a 30 minute incubation at 42° C. The resulting cDNA was analyzed by electrophoresis on a 8% sequencing gel and the size of the primer extended product determined by an end-labeled φ×174 Hinf 1 DNA-marker.

### Example 9

### Northern Hybridization

In order to verify that the shrimp act5C gene obtained was cytoplasmic, Northern hybridization experiments were performed to study the temporal and spatial expressions of the shrimp act5C gene during the shrimp life cycle. Poly(A)⁺RNA was prepared from several developmental stages, ranging from early embryo to adult (i.e. embryos, larvae, pupae, juvenile, and adult) and from various organs and tissues (i.e. brain, eyestalk, stomach, heart, hepatopancreas, ovary, and leg muscle). Equal amounts of poly(A)⁺RNA from different developmental stages and from various organs were subjected to gel electrophoresis under denaturing conditions, transferred to nitrocellulose filters, and hybridized to ³²P-labeled shrimp act5C-cDNA under conditions that are sufficiently stringent for specificity. Similar procedures of Northern hybridization as described in Sun, “Molecular Cloning and Sequence Analysis of a cDNA Encoding a Molt-Inhibiting Hormone-like Neuropeptide from the White Shrimp Penaeus vannamei,” Mol. Mar. Biol. Biotechnol. 3(1):1-6 (1994), which is hereby incorporated by reference in its entirety, were used.

Total RNA from each shrimp sample was isolated according to the method of Chomczynski et al., “Single-Step Method of RNA Isolation by Acid Guanidinium Thiocyanate-Phenol-Chloroform Extraction,” Anal. Biochem. 162(1):156-159 (1987), which is hereby incorporated by reference in its entirety, and Poly(A⁺) RNA will be obtained using the Poly(A) Quik mRNA Purification kit (Stratagene, LA Jolla, Calif.) and spectrophotometrically quantitated. RNAs to be separated were denatured by heating for 15 minutes at 65° C. One μg/lane was loaded on a 1.2% agarose-0.66 M formaldehyde gel (Lehrach et al., “RNA Molecular Weight Determinations by Electrophoresis Under Denaturing Conditions, a Critical Reexamination,” Biochemistry 16:4743-4751 (1977). The electrophoresis buffer consisted of 20 mM Na-MOPS (Sigma, St. Lous, Mo.), 5 mM NaOAc, 1 mM EDTA. After electrophoresis, the gel was blotted to a nylon membrane (Amersham, Piscataway, N.J.) in 10×SSPE. After blotting for 20 hours, filters were air dried, then baked for 2 hours in a vacuum oven.

Filters were pre-hybridized at 50° C. for 4 hours in a solution containing 50% (v/v) deionized formamide, 6×SSPE, 5× Denhardt's reagent, 0.5% SDS, and 100 μg/ml denatured salmon sperm DNA, then hybridized to the random primed labeled ³²P-act5C-cDNA in the buffer above at 50° C. for 20 hours. After hybridization, filters were washed twice at room temperature in 2×SSPE, 0.5% SDS, twice at 75° C. in 0.2×SSPE, 0.05% SDS, and exposed to Kodak XAR-5 X-ray film plus intensifying screens at −80° C.

### Example 10

### Transient Gene Expression

Transient gene expression of the EGFP gene in transgenic shrimp was monitored by fluorescent microscope examination. Due to the spectral properties of EGFP which absorbs blue light and emits green light, the expression of the EGFP can be visualized by placing the live shrimp on a dark disk under a fluorescence microscope (Leitz) adapted with a filter set (excitation wavelength of 490 nm and emission wavelength of 525 nm). The intensity of the fluorescence correlated to the EGFP level can be documented by photography.

The survival rate and the number of fluorescent eggs were determined, and the results from different promoter-regions constructs, from different animal handling conditions, and from the controls were compared.

### Example 11

### Measurement of Endogenous Fluorescence in Shrimp via Fluorescence Microscopy

Fluorescence microscopy images using fluorescein and rhodamine filter sets illustrated the relatively high levels of visible, endogenous fluorescence in the hepatopancreas and proximal regions of the animals. Furthermore, the endogenous fluorescence appears to increase as the animal matures. In order to determine whether EGFP fluorescence could be detected against the background of endogenous fluorescence in the shrimp, spectrofluorometric measurements were taken. Fluorescence microscopy was performed on live, whole shrimp at each of four developmental stages (egg, protozoea, mysis, and postlarvae) using a fluorescence inverted microscope (Zeiss Axiovert 10). Images of each shrimp viewed under brightfield, fluorescein filter set (excitation: 320-500 nm, emission: 505-560 nm) and rhodamine filter set (excitation: 370-590 nm, emission: 570-600 nm), were captured with a 12-bit CCD camera system (Photometrics). These shrimp were also photographed with a digital camera (Nikon) mounted to a stereo dissection microscope (Zeiss, Jena, Germany).

### Example 12

### Monitoring EGFP and DsRed Reporter Gene Expression via Spectrofluorometry

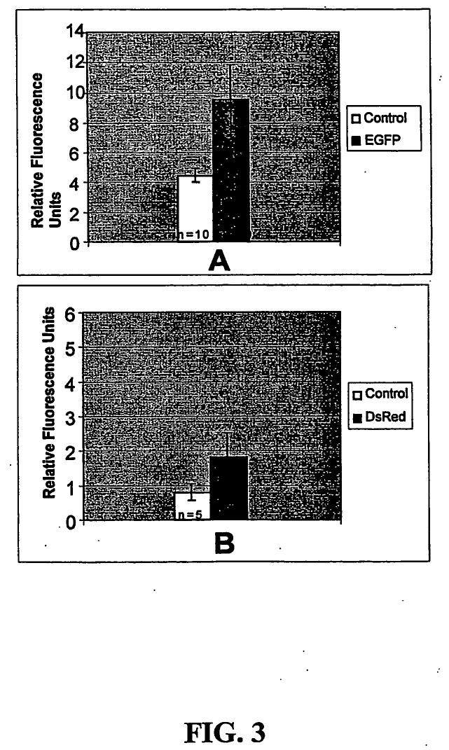

Plasmid DNA consisting of 4.5 μg of the vector EGFP-N1, in 2 μl 10 mM Tris, pH 7.8, was injected into juvenile shrimp (4 cm in length) at the second abdominal muscle segment under the exoskeleton. This procedure was repeated using the vector, 2-N1 (Clontech Laboratories, Inc., Palo Alto, Calif.). The DsRed vector encodes the red fluorescent protein from Discosoma sp. Two days after injection, injected tissue segments were excised from shrimp and homogenized. Fluorescent intensity of the homogenate supernatant was measured using a fluorescence spectrophotometer (F-2500, Hitachi) at appropriate wavelengths (excitation: 488 nm, emission: 507 nm for EGFP; and excitation: 558 nm, emission: 583 nm for DsRed). Results from the analysis of EGFP and DsRed expression efficiency in shrimp via muscular injection, is shown in FIGS. 3A-B. Expression of both EGFP and DsRed are approximately 2 times higher than fluorescence of the controls, demonstrating their suitability as a marker gene. However, EGFP may be preferable to DsRed as a marker gene since greater variability and inaccuracy may be associated with DsRed's low fluorescent intensity values and the extended protein maturation time (˜20 hrs).

### Example 13

### Gene Integration and Expression

The expression of the GFP gene in the egg, larva, and juvenile were followed by fluorescent microscopy as described above, and also by spectrofluorescent measurement. The GFP in the protein extract was quantified by measuring emission at 509 nm when exited at 395 nm using a spectrofluorometer (Kratos FS 970). Fluorescence intensity was normalized to protein concentration as determined by Bradford assay using the Bio-Rad protein assay kit (Bio-Rad Lab, Hercules, Calif.). For Southern hybridization, genomic DNA was isolated from the control and putative transformed shrimp using Easy DNA kit (Invitrogen, Carlsbad, Calif.). After digestion with appropriate restriction enzyme(s), the DNA was subjected to gel electrophoresis, transferred to a Nylon membrane, and fixed with UV light cross-linking. Blots were hybridized with the GFP DNA fragment labeled with Digoxigenin as described by Sun, “Expression of the Molt-Inhibiting Hormone-like Gene in the Eyestalk and Brain of the White Shrimp Penaeus vannamei,” Mol. Mar. Biol. Biotechnol. 4(3):262-268 (1995), which is hereby incorporated by reference in its entirety. Genomic DNAs isolated from the transgenic animals were used as templates for polymerase chain reaction assay (Sun, “Recombinant Molt-Inhibiting Hormone-like Neuropeptide Produced in the Yeast Pichia pastoris,” In: PACON International Proceedings. Aug. 5-8, 1997, Hong Kong, pp. 509-518 (1997), which is hereby incorporated by reference in its entirety) to confirm the GFP gene has integrated into the shrimp genome. To detect and localize the GFP transcripts in the transgenic shrimp, techniques of SDS-polyacrylamide gel electrophoresis, Western blot analysis and in situ hybridization of the shrimp tissue sections were performed Sun, “Expression of the Molt-Inhibiting Hormone-like Gene in the Eyestalk and Brain of the White Shrimp Penaeus vannamei,” Mol. Mar. Biol. Biotechnol. 4(3):262-268 (1995), Sun, “Recombinant Molt-Inhibiting Hormone-like Neuropeptide Produced in the Yeast Pichia pastoris,” In: PACON International Proceedings. Aug. 5-8, 1997, Hong Kong, pp. 509-518 (1997), which are hereby incorporated by reference in their entirety).

### Example 14

### Identification and Characterization of a Gene-Specific DNA Fragment Encoding the Cytoplasmic β-actin from Shrimp, L. vannamei

Actin is a major protein constituent of all eukaryotic cells. In vertebrates at least six actin variants have been characterized: two from smooth muscles, two from striated muscles, and two from non-muscle tissues (β and γ) (Vandekerckhove et al., “The Complete Amino Acid Sequence of Actins from Bovine Aorta, Bovine Heart, Bovine Fast Skeletal Muscle, and Rabbit Slow Skeletal Muscle. A Protein-Chemical Analysis of MuscleActin Differentiation,” Differentiation 14(3):123-133 (1979), which are hereby incorporated by reference in their entirety). Although the actin gene family is expressed in all tissues, individual actin genes show tissue and developmental specificity in their expression (Fyrberg et al., “Transcripts of the Six Drosophila Actin Genes Accumulate in a Stage-and Tissue-Specific Manner,” Cell 33(1):115-123 (1983); Sanchez et al., “Two Drosophila actin Genes in Detail: Gene Structure, Protein Structure, and Transcription During Development,” J. Mol. Biol. 163:533-551 (1983); Vandekerckhove et al., “Chordate Muscle Actins Differ Distinctly from Invertebrate Muscle Actins. The Evolution of the Different Vertebrate Muscle Actins,” J. Mol. Biol. 179(3):391-413 (1984), which are hereby incorporated by reference in their entirety). There are also six actin genes found in the invertebrate fly, Drosophila melanogaster. Two of the Drosophila actin genes, act5C and act42A, are expressed in undifferentiated cells and encode cytoplasmic or non-muscle actins (Fyrberg et al., “Transcripts of the Six Drosophila Actin Genes Accumulate in a Stage-and Tissue-Specific Manner,” Cell 33(1):115-123 (1983), which is hereby incorporated by reference in its entirety). The remaining four genes probably respond to regulatory molecules and are synthesized during early muscle cell differentiation. These invertebrate cytoplasmic actin genes are different from vertebrate non-muscle actin genes in terms of amino acid sequences and isoelectric points of the protein molecules. However, β-actin is the major non-muscle or cytoplasmic actin isoform and it is expressed in most eukaryotic non-muscle cells, as well as in undifferentiated myoblasts. And, because β-actin promoter is an active cellular promoter (Gunning et al., “A Human β-Actin Expression Vector System Directs High-Level Accumulation of Antisense Transcripts,” Proc. Natl. Acad. Sci. USA 84:4831-4835 (1987), which is hereby incorporated by reference in its entirety) and has constitutive expression properties, β-actin gene(s) are a prime target for transgenic manipulation technology.

Reverse-transcriptase-polymerase chain reaction (RT-PCR) method (Sun, “Expression of the Molt-Inhibiting Hormone-like Gene in the Eyestalk and Brain of the White Shrimp Penaeus vannamei,” Mol. Mar. Biol. Biotechnol. 4(3):262-268 (1995)) was used to generate a DNA fragment encoding the partial β-actin5C from shrimp tissue, as described in Example 4. Degenerate primer pairs P1-P2, described in Example 4, were constructed against conserved regions of the fruit fly, Drosophila melanogaster actin5C protein (Bond et al., “The Drosophila Melanogaster Actin 5C Gene Uses Two Transcription Initiation Sites and Three Polyadenylation Sites to Express Multiple mRNA Species,” Mol. Cell Biol. 6(6):2080-2088 (1986), which are hereby incorporated by reference in their entirety). Total RNA isolated from shrimp tissue using the method described by Chomczynski et al., “Single-Step Method of RNA Isolation by Acid Guanidinium Thiocyanate-Phenol-Chloroform Extraction,” Anal. Biochem. 162(1):156-159 (1987), which is hereby incorporated by reference in its entirety, was used as template for the RT-PCR reaction. Size analysis of RT-PCR products by ethidium bromide-agarose gel electrophoresis revealed a high intensity DNA band of about 224 bp, which was the size expected using the P1/P2 primer set. The PCR-generated DNA product was purified from the agarose gel, cloned with a pCR2.1 vector using the original TA cloning kit (Invitrogen, Carlsbad, Calif.). The PCR-generated 224-bp DNA fragment encoding the shrimp β-actin5C was amplified, purified and labeled using the methods described by Sun (Sun, “Molecular Cloning and Sequence Analysis of a cDNA Encoding a Molt-Inhibiting Hormone-like Neuropeptide from the White Shrimp Penaeus vannamei,” Mol. Mar. Biol. Biotechnol. 3(1):1-6 (1994), Sun, “Expression of the Molt-Inhibiting Hormone-like Gene in the Eyestalk and Brain of the White Shrimp Penaeus vannamei,” Mol. Mar. Biol. Biotechnol. 4(3):262-268 (1995), which are hereby incorporated by reference in their entirety), and used as an effective probe to identify the full-length cDNA and the genomic clones of the shrimp β-actin5C gene by screening the existing cDNA and genomic libraries of the shrimp L. vannamei. The full-length genomic DNA sequence of the shrimp β-actin gene was generated via PCR using primers whose sequences were based on a region of the promoter and the 3′ untranslated region of the shrimp β-actin cDNA. The PCR product generated was then used as a template in a 2^{nd} round of the PCR using nested primers whose sequences were based on the shrimp β-actin cDNA.

### Example 15

### Species Comparison of the Partial Amino Acid Sequence of Shrimp β-actin5C

In comparing the partial deduced amino acid sequence of the shrimp β-actin5C with other cytoplasmic β-actin proteins from other species, it was found that the shrimp β-actin5C shares more than 90% homology in amino acid sequences studied with crab (C. carnifex), fly (D. Melanogaster), nematode (C. elegans), and chicken. It was also noted that a set of eleven amino acid residues in the shrimp β-actin5C is found missing at position #24 to position #34; and an additional amino acid, aspartic acid, is present at position #59.

### Example 16

### Spatial Expression of the Partial Shrimp β-actin5C DNA

Transcripts from the partial β-actin5C gene are found in most of the shrimp system including eye, stomach, heart, and hepatopancreas when using the RT-PCR technique for the detection. Expression of the shrimp β-actin5C gene is especially abundant in hepatopancreas but no expression was found in muscle. This observation suggests that the shrimp β-actin5C transcript is present in organs of non-muscle type and is thought to be a cytoplasmic form of actin.

### Example 17

### Construction and Screening of the Shrimp Genomic Library

A genomic library of the Pacific white shrimp L. vannamei (1.2×10⁶ recombinants) was constructed with the LambdaGEM-11 vector (Promega, Madison, Wis.) using genomic DNA prepared by Easy DNA kit (Invitrogen, Carlsbad, Calif.). The purified genomic DNA was partially digested by Sau3A and fragments of 15-23 kb were ligated into the LambdaGEM-11 vector. Packaging was performed using the Packagene Extract system (Promega, Madison, Wis.).

Approximately 1×10⁴ plaques were screened by a combination of PCR amplification method (Amaravadi et al., “A Rapid and Efficient, Nonradioactive Method for Screening Recombinant DNA Libraries,” Biotechniques 16(1):98-103 (1994), which is hereby incorporated by reference in its entirety) and the in situ plaque hybridization technique (Benton et al., “Screening Lambda gt Recombinant Clones by Hybridization to Single Plaques in situ,” Science 196(4286):180-182 (1977), which is hereby incorporated by reference in its entirety) from the shrimp genomic library. Primers were made based on the 224-bp DNA sequence (see Example 14) for the PCR assay. The PCR-generated 224-bp DNA fragment was used as a probe for in situ plaque hybridization. A total of twelve positive clones were isolated. The positive genomic clones were grown and the bacteriophage DNAs were prepared by using λ-DNA purification kit (Stratagene, La Jolla, Calif.). Purified phage DNA were analyzed on Southern blot. The sizes of phage DNA as revealed by ethidium bromide staining and UV illumination after agarose gel electrophoresis was ranged from 1.0 to 18 kd. The positive restriction enzymes digested fragments were selected and subcloned into the Bluescript vector (Stratagene, La Jolla, Calif.) for DNA sequencing and analysis. These DNA samples were then processed for DNA sequencing and assembling.

### Example 18

### β-Actin Promoter Sequence Identification

Using the gene walking method, a 1297-bp promoter of the shrimp β-actin gene was identified and sequenced. This promoter contains a CAAT box, TATA box, and CArG sequence that are characteristic of β-actin promoters found in other organisms. This promoter, termed β-ActinP2, identified herein as having SEQ ID NO: 1, was cloned and used in vector construction. A full-length cDNA encoding the β-actin (Genbank Accession No. AF300705) and its promoter sequence from the Pacific white shrimp L. vannantei was also identified, cloned, and sequenced (Genbank Accession No. AF300705). The cDNA for β-Actin is identified herein as SEQ ID NO: 2.

### Example 19

### Actin Promoter Sequence Identification

The existing shrimp genomic library was screened for the genomic clone of actin using a combination of PCR amplification method (Amaravadi et al., “A Rapid and Efficient Nonradioactive Method for Screening Recombinant DNA Libraries,” Biotechniques 16(1):98-103 (1994), which is hereby incorporated by reference in its entirety) and the in situ plaque hybridization technique (Benton et al., “Screening Lambdagt Recombinant Clones by Hybridization to Single Plaques in situ,” Science 196(4286):180-182 (1977), which is hereby incorporated by reference in its entirety). The PCR-generated 224-bp DNA fragment (See Example 14) labeled with digoxigenin was used as a probe for non-radioactive in situ plaque hybridization. The positive genomic clones were isolated, grown, and the bacteriophage DNAs were prepared using λ-DNA purification kit (Qiagen, Inc., Valencia, Calif.). The positive restriction enzyme digested fragments were selected and subcloned into the Bluescript vector (Stratagene, La Jolla, Calif.) for DNA sequencing and analysis. These DNA samples were then processed for DNA sequencing and assembling.

The shrimp actin promoter (SEQ ID NO: 4) contains TATA and CAAT boxes approximately 500 base pairs upstream from the translation start site. Unique CACA-rich and CATA-rich regions are located in the actin promoter region upstream from the expected TATA and CAAT boxes. The deduced polypeptide of the shrimp actin consists of a 64-amino acid signal peptide and a 311-amino acid mature polypeptide. This shrimp actin exhibits 94% amino acid homology with the tiger prawn (Penaeus monodon) actin, 93% homology with the rattail fish (Coryphaenoides acrolepis) skeletal alpha actin type 2, and 93% homology with human (Homo sapiens) alpha actin of the cardiac muscle.

### Example 20

### Actin cDNA Sequence Identification

The deduced polypeptide of the shrimp actin consists of a 64-amino acid signal peptide and a 311-amino acid mature polypeptide. This shrimp actin exhibits 94% amino acid homology with the tiger prawn (Penaeus monodon) actin, 93% homology with the rattail fish (Coryphaenoides acrolepis) skeletal alpha actin type 2, and 93% homology with human (Homo sapiens) alpha actin of the cardiac muscle.

### Example 21

### Expression of a Reporter Gene in Shrimp Muscle by Injection

In order to test the ability of a heterologous promoter to drive expression of a reporter gene and to investigate parameters of introducing exogenous DNA into shrimp system, a trial experiment was performed in which an expression vector containing a promoter of human cytomegalovirus (CMV) sequence and a reporter gene of β-galactosidase (β-Gal) was prepared and delivered into shrimp muscle via direct injection. Injection was performed with a 33-gauge hypodermic needle filled with various amount of super-coiled plasmid DNA in 2.5 ul Pantin's saline buffer (Pantin, 1934, which is hereby incorporated by reference in its entirety) into the fourth tail segment of juvenile white shrimp (approximately 5-6 inches in length). The expression efficiency was monitored spectrophotometrically using the β-Galactosidase enzyme assay system (Promega, Madison, Wis.). Muscle biopsy samples were taken for determining the level of expression at day one through day ten after plasmid DNA injection. Control muscle samples from shrimp injected with Pantin's saline buffer alone were also taken. Most of the samples from shrimp injected with the pCWV-β-Gal showed β-Gal activity upon assay, whereas no significant activity was observed in control samples. The survival rate was found to be 95% in a total of 76 animals tested. Expression of the reporter gene as monitored spectrophotometrically was observed 24 hours after injection with highest expression at day two. The exogenous DNA of β-galactosidase was detected by polymerase chain reaction four days after injection. These results demonstrated that micro-injection into shrimp muscle is a potential technique for testing transient expression of foreign gene in shrimp system.

### Example 22

### Generating DNA Fragments Encoding the TSV-CP by RT-PCR

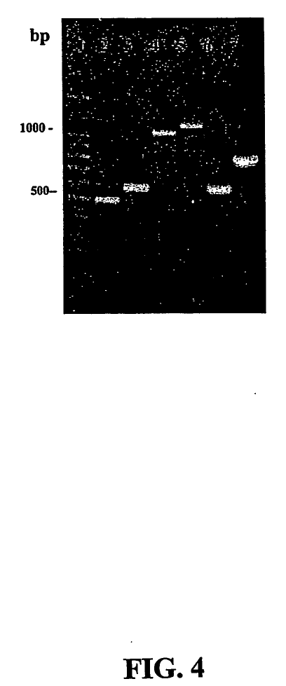

The genomic organization of TSV consists of a linear, positive-sense, single stranded RNA of approximately 9 kb in length. Its capsid consists of three major polypeptides (24, 40, and 55 Kd) and one minor polypeptide (58 Kd) (Mari et al., “Full Nucleotide Sequence and Genome Organization of the Taura Syndrome Virus of Penaeid Shrimp,” Unpublished (2000); Genbank Accession Number: AF277675, which are hereby incorporated by reference in their entirety). One of the genes encoded by the RNA is a 111 Kd viral coat protein (Genbank Accession #AF277378, which is hereby incorporated by reference in its entirety). This coat protein is most likely cleaved co- and post-translationally since the proteinic capsid of purified TSV was found to consist of three major (55, 40, and 24 Kd) polypeptides and one minor (58 Kd) polypeptide (Bonami et al., “Taura Syndrome of Marine Penaeid Shrimp: Characterization of the Viral Agent,” J. Gen. Virol. 78:313-319 (1997), which is hereby incorporated by reference in its entirety). This gene encoding the structural coat protein was selected as a prime candidate for developing of viral protection in shrimp. Total RNA was isolated from TSV-infected shrimp. Several gene specific oligonucleotide primers were synthesized based on the published TSV coat protein (TSV-CP) gene sequence (Genbank Accession No. AF277378, which is hereby incorporated by reference in its entirety). Use of these gene specific primers and TSV RNA in the RT-PCR assay yielded distinct, high-intensity bands, corresponding to the expected sizes, as shown in FIG. 4. These DNA fragments were purified, cloned, sequenced, and various fragments from about 500 to 1000 bp of the TSV coat protein gene in both positive and negative orientations were used in vectors constructed for transfer into shrimp.

### Example 23

### Generating DNA Fragments Encoding the IHHNV-CP by RT-PCR

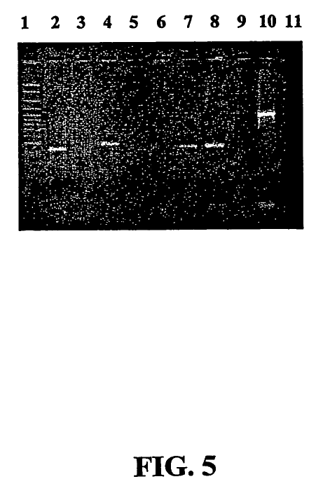

IHHNV is a single-strand DNA virus with a viral coat protein of 37.5 Kd (Genbank Accession #AF218266, which is hereby incorporated by reference in its entirety). Eight oligonucleotides, gene specific to the IHHNV, were synthesized (Biotechnology/Molecular Biology Instrumentation Facility, University of Hawaii) based on the published nucleotide sequences of the IHHNV gene and were used as primers in the RT-PCR assays. Shrimp samples infected with IHHNV were obtained from Dee Montgomery-Brock (Aquaculture Development Program, Department of Agriculture, State of Hawaii). Approximately 0.25 g of the muscle tissue were ground into powder in liquid nitrogen and total RNA was isolated using the Purescript RNA isolation kit (Gentra Systems, Inc.), and used as template in RT-PCR. The RT-PCR assays were performed according to the procedures described by Sun (Sun, “Expression of the Molt-Inhibiting Hormone-like Gene in the Eyestalk and Brain of the White Shrimp Penaeus vannamei,” Mol. Mar. Biol. Biotechnol. 4(3):262-268 (1995), which is hereby incorporated by reference in its entirety) using the GeneAmp RNA PCR Kit (PE Biosystems, Foster City, Calif.). Several DNA bands were generated via RT-PCR assays using the synthesized primers and the IHHNV-RNA as template. Results are summarized in Table 1, below, and shown in FIG. 5.

TABLE 1

IHHNV Gene Specific Oligonucleotide Primers

| | |
|---|---|
| SEQ | DNA size |

| | | | | |
|---|---|---|---|---|
| | ID | | | Gener- |
| Primer | NO: | Sequence | Expected | ated |

| | | | | |
|---|---|---|---|---|
| 1 | 9 | 5′-CAA ACT ATG AAG ACC CAA | 431 bp | 431 bp |
| | | TCC-3′ | | |
| | 10 | 5′-ATA TTT AGT TAG TAT GCA | | |
| | | TA-3′ | | |
| 2 | 11 | 5′- | 1021 bp | none |
| | | AAGGAAATCGACGACATCATA-3′ | | |
| | 12 | 5′-ATATTTAGTTAGTATGCATA-3′ | | |
| 3 | 13 | 5′- | 471 bp | 471 bp |
| | | AAGGAAATCGACGACATCATA-3′ | | |
| | 14 | 5′-ATA TGG GGT GTC TGT AAA | | |
| | | TGT G-3′ | | |
| 4 | 15 | 5′-ATG TGC GCC GAT TCA ACA | 1019 bp | none |
| | | A-3′ | | |
| | 16 | 5′-AAT CGG GTA TAT ATT GCA | | |
| | | CA-3′ | | |
| 5 | 17 | 5′-ATG TGC GCC GAT TCA ACA | 993 bp | none |
| | | A-3′ | | |
| | 18 | 5′-ATA TTT AGT TAG TAT GCA | | |
| | | TA-3′ | | |
| 6 | 19 | 5′-ATG TGC GCC GAT TCA ACA | 443 bp | 443 bp |
| | | A-3′ | | |
| | 20 | 5′-ATA TGG GGT GTC TGT AAA | | |
| | | TGT G-3′ | | |
| 7 | 21 | 5′-CAA ACT ATG AAG ACC CAA | 457 bp | 457 bp |
| | | TCC-3′ | | |
| | 22 | 5′-AAT CGG GTA TAT ATT GCA | | |
| | | CA-3′ | | |
| 8 | 23 | 5′- | 1047 bp | 1047 bp |
| | | AAGGAAATCGACGACATCATA-3′ | | |
| | 24 | 5′-AATCGGGTATATATTGCACA-3′ | | |

DNA fragments of about 400 to 500 bp of the IHHNV coat protein gene in sense and anti-sense orientations for vector construction were used to develop plasmid constructs for transfer into shrimp.

### Example 24

### Detection Methods for TSV and IHHNV

Detection of TSV using sequence information from the cDNA segment of the TSV genome and the reverse transcription-polymerase chain reaction (RT-PCR) assay has been developed (Nunan et al., “Reverse Transcription Polymerase Chain Reaction (RT-PCR) Used for the Detection of Taura Syndrome Virus (TSV) in Experimentally Infected Shrimp,” Dis. Aquatic. Org. 34:87-91 (1998), which is hereby incorporated by reference in its entirety) and used widely for monitoring TSV infection in farmed shrimps.

Detection and quantification of IHHNV using real-time polymerase chain reaction (PCR) has been developed (Tang et al., “Detection and Quantification of Infectious Hypodermal and Hematopoietic Necrosis Virus in Penaeid Shrimp by Real-Time PCR,” Dis. Aquat. Org. 44(2):79-85 (2001); Dhar et al., “Quantitative Assay for Measuring the Taura Syndrome Virus and Yellow Head Virus Load in Shrimp by Real-Time RT-PCR Using SYBR Green Chemistry,” J. Virol. Methods. 104(1):69-82 (2002), which are hereby incorporated by reference in their entirety) and is considered a rapid and highly sensitive method for IHHNV detection in shrimp. Identification of genetic markers as predictors for IHHNV resistance in shrimp have also been reported (Hizer et al., “RAPD Markers as Predictors of Infectious Hypodermal and Hematopoietic Necrosis Virus (IHHNV Resistance in Shrimp (Litopenaeus Stylirostris),” Genome/Natl. Res. Council Canada 45(1):1-7 (2002), which is hereby incorporated by reference in its entirety).

### Example 25

### Expression Vector Construction

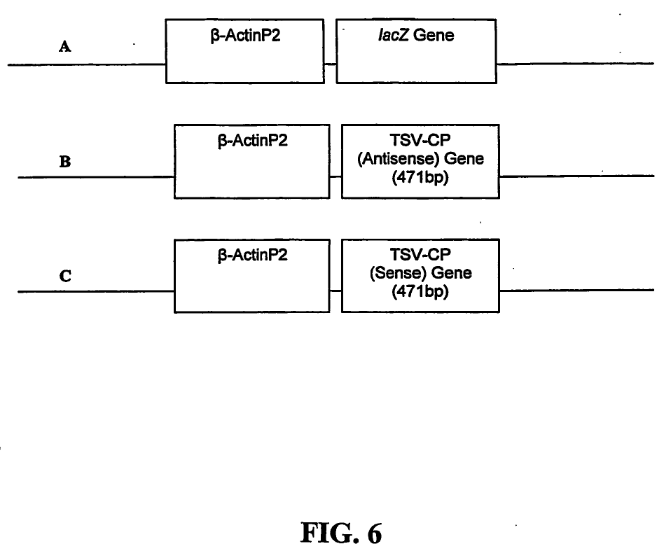

Expression vectors were constructed consisting of the chimeric shrimp β-actin promoter, a sense (5′→3′) or antisense (3′→5′) oriented fragment of the TSV-CP target gene, or a reporter gene. The pSV-β-Galactosidase vector (Promega, Madison, Wis.) or pEGFP-N1 (Clontech, Palo Alto, Calif.) were used as the base vectors. A series of vectors as constructed are shown in FIGS. 6A-C. Using PCR methodology, NcoI and Hind III restriction enzyme sites were created at the 5′ end and 3′ end, respectively, of the β-actin promoter of the present invention, β-ActinP2. The SV40 promoter and enhancer of the pSV-β-Galactosidase (β-Gal) vector were excised through restriction enzyme digestion with NcoI and Hind III, and the β-ActinP2 was inserted into the vector to construct the expression vector, pβ-ActinP2-β-Gal, shown in FIG. 6A. In addition, Hind III and Sal I restriction enzyme sites were added to the 471-bp TSV-CP target gene using PCR. The lacZ gene of the pβ-ActinP2-β-Gal vector was replaced with the TSV-CP target gene in antisense orientation by restriction enzyme digestion with Hind III and SalI to produce the expression vector, pβ-ActinP2-TSV-CP-AS (471 bp), as shown in FIG. 6B. A third expression vector, pβ-ActinP2-TSV-CP-S (471 bp) was constructed with the TSV-CP target gene in the sense orientation as shown in FIG. 6C. The expression vectors were cloned, purified, and introduced into shrimp embryos through electroporation and microinjection.

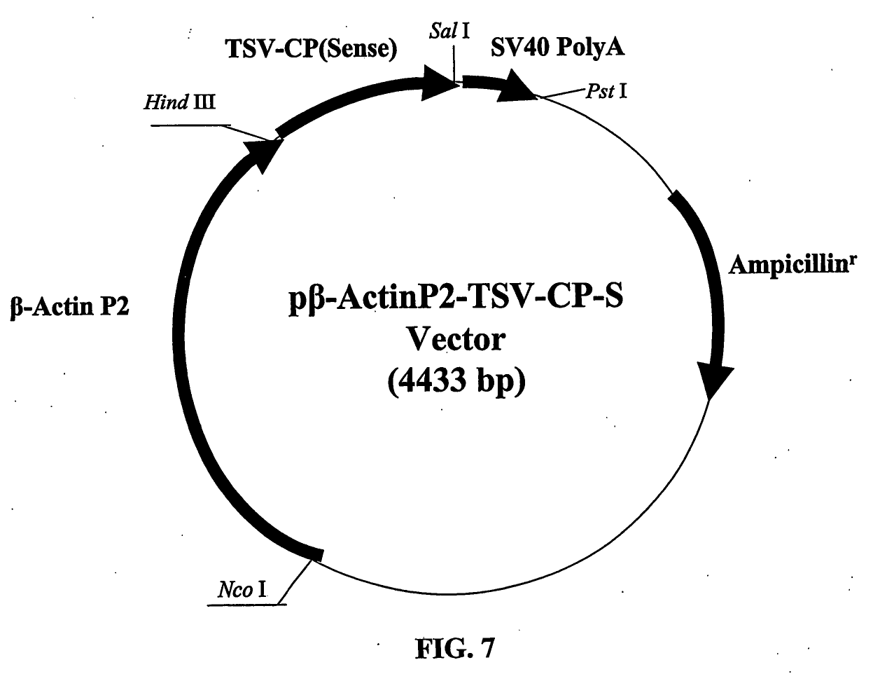

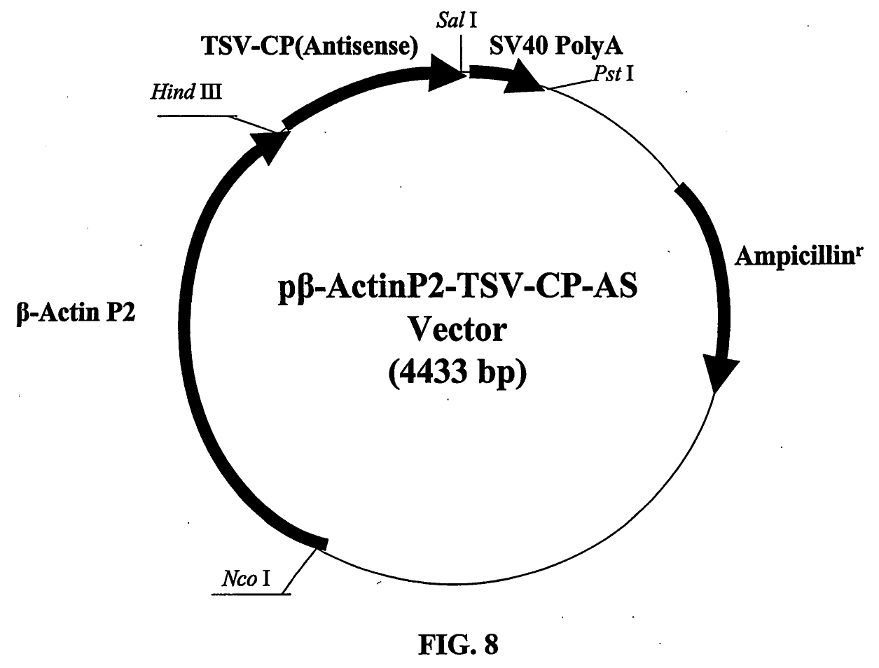

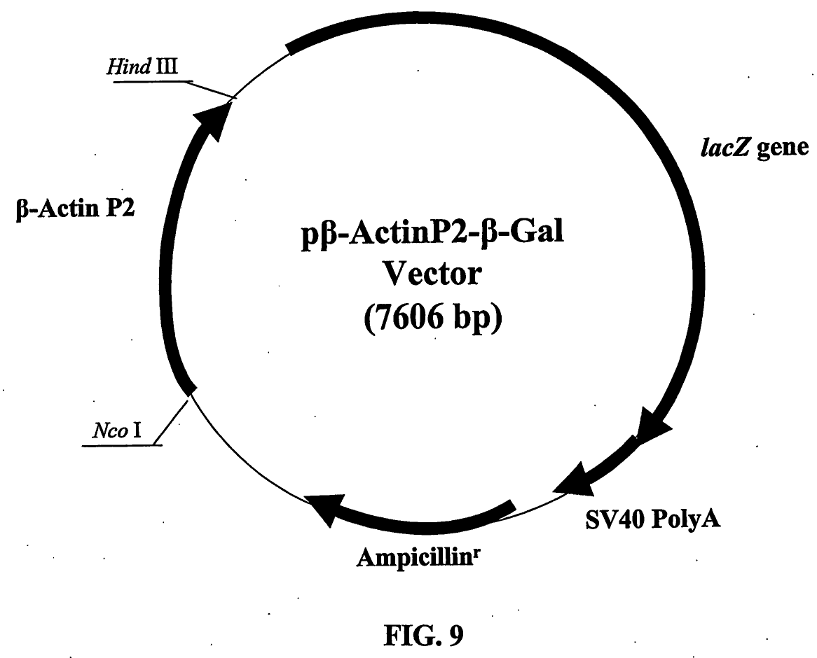

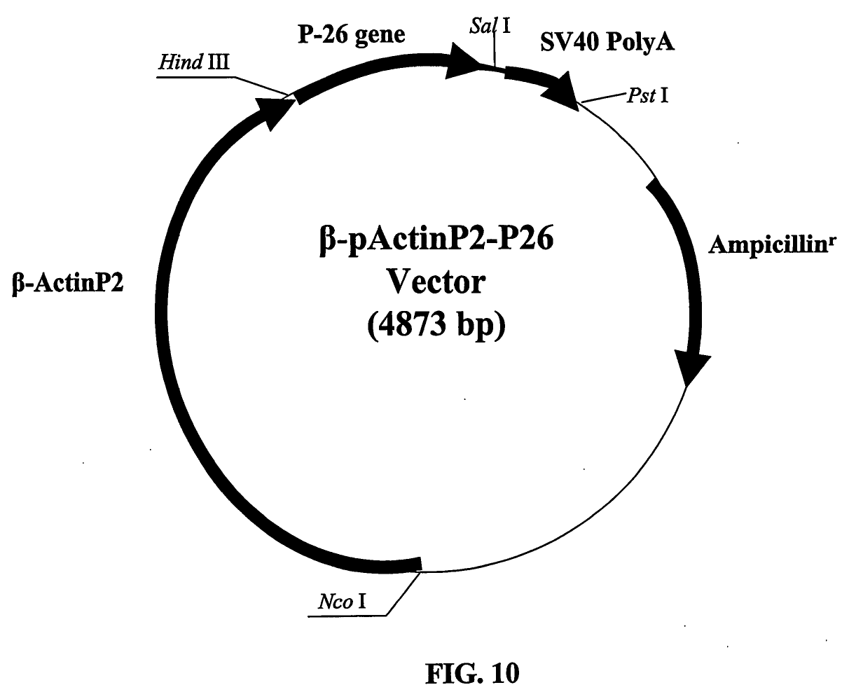

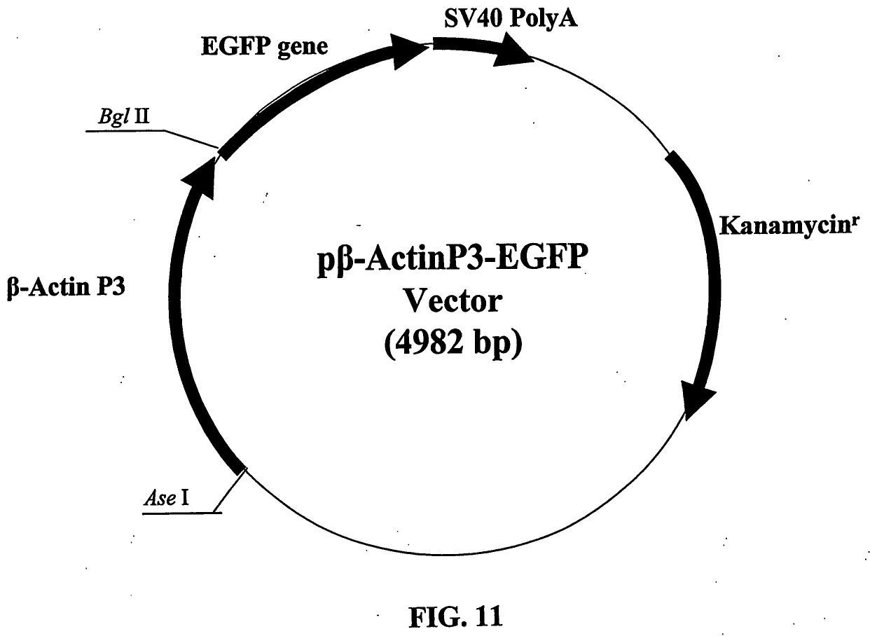

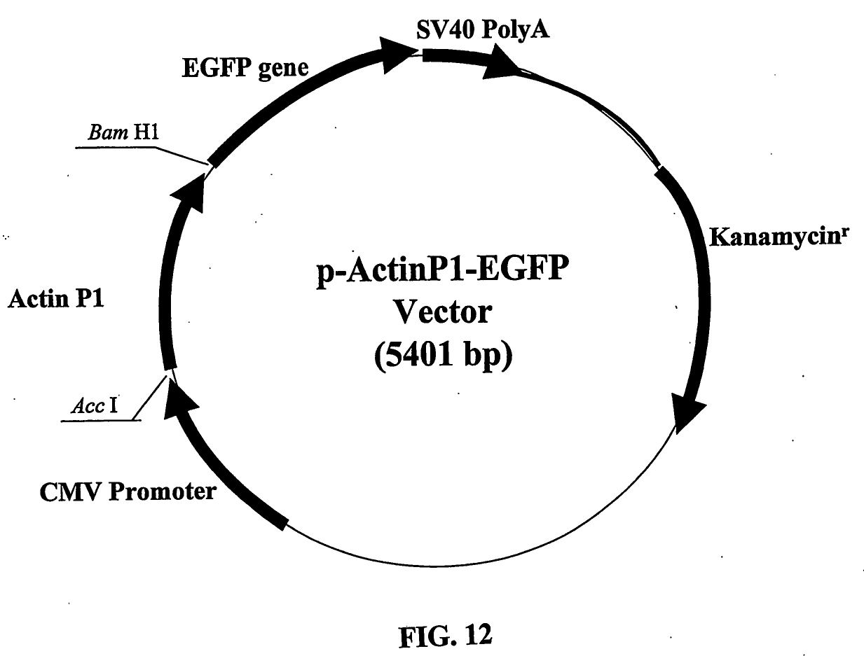

A brief description and plasmid map of these and other vector constructs are provided as follows. FIG. 7 shows the pβ-ActinP2-TSV-CP-S vector which contains the 1234-bp promoter region (β-ActinP2) of the shrimp β-actin gene. The target gene of this vector is the 491-bp Taura syndrome virus coat protein (TSV-CP) fragment in sense orientation. FIG. 8 shows the pβ-ActinP2-TSV-CP-AS vector containing the 1234-bp promoter region (β-ActinP2) of the shrimp β-actin gene. The target gene of this vector is the 491-bp TSV-CP fragment in antisense orientation. FIG. 9 shows the pβ-ActinP2-β-Gal vector containing the 1234-bp promoter region (β-ActinP2) of the shrimp β-actin gene. This vector contains the 3301-bp lac Z gene for β-Gal as the reporter gene. FIG. 10 shows the ActinP2-P26 vector containing the 1234-bp promoter region of the shrimp β-actin gene. The target gene of this vector is the 791-bp heat shock protein 26 (P26) gene from the brine shrimp, Artemia franciscana. FIG. 11 shows the pβ-ActinP3-EGFP vector containing the 893-bp promoter region (β-ActinP3) of the shrimp beta-actin gene. The 718-bp enhanced green fluorescent protein (EGFP) gene from the jellyfish is the reporter gene in this vector. FIG. 12 shows the p-ActinP1-EGFP vector containing a 721-bp fragment of the promoter region (ActinP1) of the shrimp actin gene. The 718-bp enhanced green fluorescent protein (EGFP) gene from the jellyfish is the reporter gene in this vector.

### Example 26

### Delivery of Expression Vectors into Shrimp Embryos by Electroporation

Electroporation experiments were carried out with an Electro Square Porator ECM 830 (BTX). Optimal conditions for obtaining the highest hatching rate of the shrimp eggs were examined by adjusting variable parameters including voltage, electroporation pulse-length, and number of pulses. In a trial experiment, the Petri Pulser PP35-2P model was used. Circular plasmid DNA was dissolved in 0.77 M mannitol in a total volume of 2 ml at a concentration of 35 μg/ml. Fertilized eggs were de-coated with a buffer containing 32 g NaCl, 0.8 g KCl, 0.36 g NaHCO₃, and 0.28 g NaH₂PO₄ in one liter of distilled water, pH=7.4 or with 0.1 mM of 3-amino-1,2,4 triazole (ATA) prior to electroporation. About 400 fertilized shrimp eggs were placed in the petri dish (35×10 mm) containing the DNA/mannitol solution. After the electric pulse, the eggs were returned to clean sea water (28° C.) with aeration. The hatching rate was recorded and compared from each electroporation setting. The optimal settings which provided the highest hatching rate of 35% were found to be: field strength of 40 V/cm; pulse length of 10 us; and 15 pulses.

### Example 27

### Effect of Temperature on the Survival of L. vannamei upon TSV Infection

L. vannamei exposed to TSV exhibited a markedly higher survival rate when reared at a temperature of 32° C. than shrimp raised at 26° C. Nineteen out of twenty shrimp survived TSV exposure when raised at water temperature of 32° C., whereas no survival was observed when TSV-exposed shrimp were raised at 26° C. TSV was detected in all of the shrimp samples from the 26° C. tank, while most of the shrimp samples from the 32° C. tank were TSV negative according to RT-PCR analysis. This study demonstrated that temperature is a factor that influences the survival rate of shrimp challenged with TSV. In light of these preliminary results, it is hypothesized that the enhanced survival rate of shrimp at 32° C. may be due to reduced viability of TSV at that temperature, or may be due to heat-activated expression of some gene which functions in the defense mechanism of shrimp. Results from a pilot experiment showed that heat shock protein 70 (HSP70) gene was detected in all TSV-infected samples.

### Example 28

### Vector Efficiencies in Shrimp

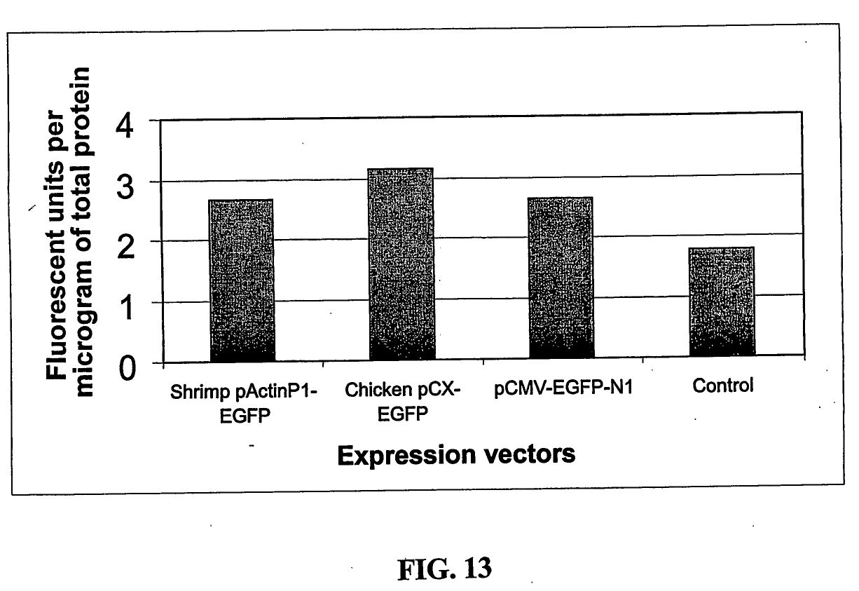

The efficiency of the shrimp pActinP1-EGFP vector was compared to the chicken pCX-EGFP vector and the pCMV-EGFP-N1 vector, as shown in FIG. 13. The vectors were introduced into shrimp via intra-muscular injection and EGFP expression was monitored by spectrofluorometer (excitation wavelength: 488 nm, emission wavelength: 507 nm). The shrimp pActinP1-EGFP vector has an EGFP expression level comparable to the pCMV-EGFP-N1 vector in the shrimp system.

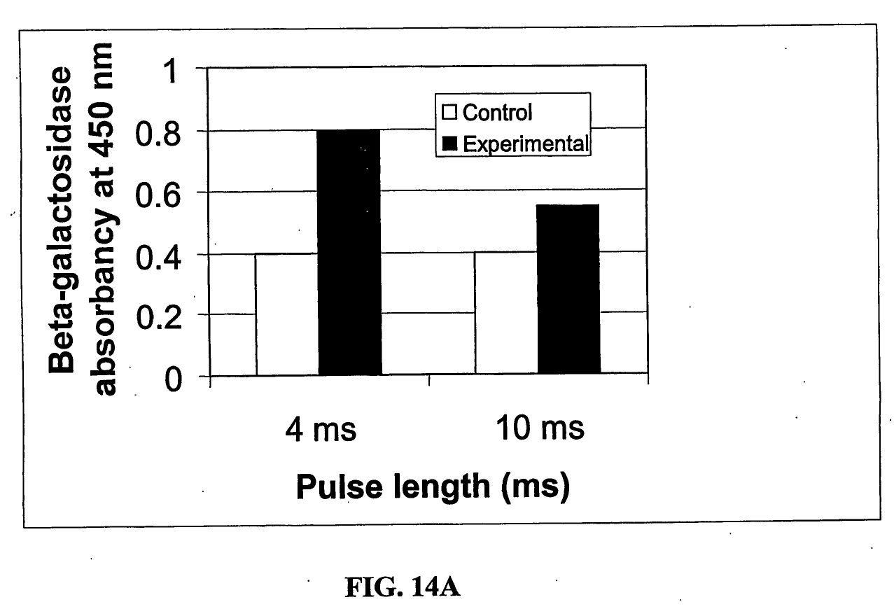

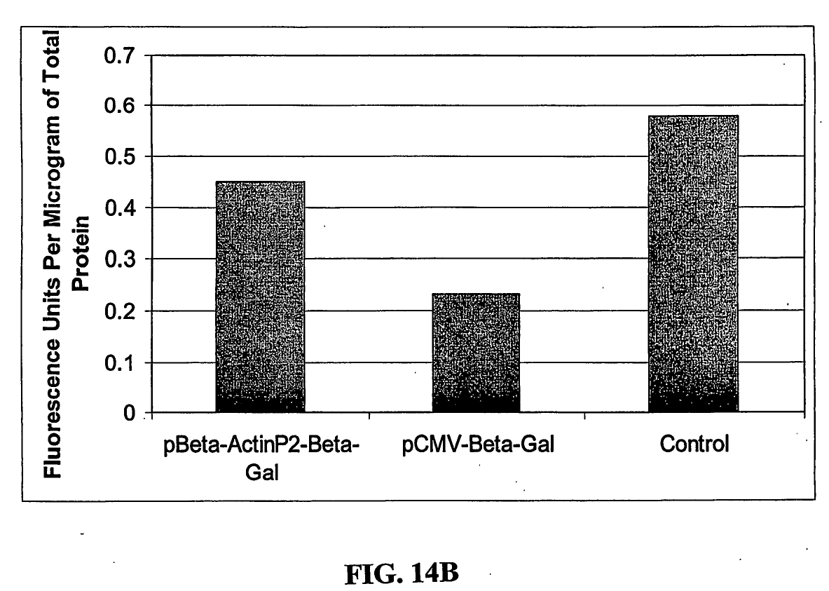

The efficiency of the shrimp pβ-ActinP2-β-Gal vector was determined through electroporation and microinjection of A. franciscana embryos. As shown in FIGS. 14A-B, the vector pβ-ActinP2-β-Gal exhibits higher beta-gal expression than the control samples.

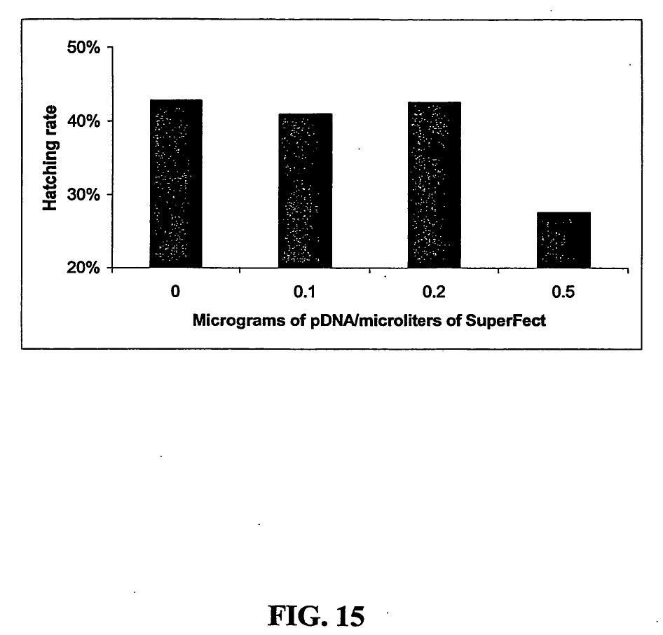

Transfection of shrimp embryos of L. vannamei via transfection reagents including SuperFect, Effectene, Jet PEI, and Lipofectamine 2000 were used to facilitate the delivery of β-ActinP2-TSV-CP-AS vector. Transfection efficiency was evaluated by both the hatching rate of shrimp embryos and transient gene expression detected through RT-PCR. Optimal DNA delivery conditions were examined by exposure of embryos to different ratios of foreign DNA and transfection reagents, in combination with electroporation, as well as different embryonic stages (from 10-50 minutes post-fertilization). Significant inhibition was observed in embryos exposed to 0.5 μg DNA/SuperFect ratio, where greater levels of free DNA were present. As shown in FIG. 15, the highest hatching rates were observed at 0.1 and 0.2 μg of DNA/μl of SuperFect (1 μl=3 μg of active SuperFect), in which the number of eggs hatched was comparable to control. In addition, manipulation of embryos prior to 18 minutes post-fertilization resulted in no hatch. Highest hatchability was observed in embryos manipulated at 35 minutes post-fertilization.

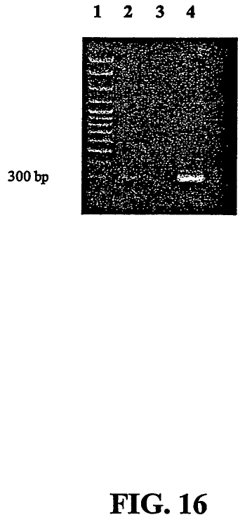

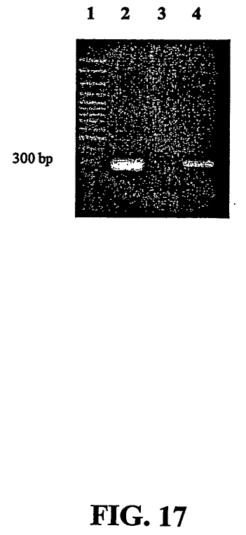

The function of the shrimp expression vectors of pβ-ActinP2-TSV-CP-S and pβ-ActinP2-TSV-CP-AS were tested by introducing the vectors into shrimp embryos via microinjection and electroporation. RT-PCR method was used to verify target gene (TSV-CP) expression in the putative transgenic shrimp at the mysis stage (day 8 after hatching, as shown in FIG. 16 and FIG. 17). Results from these two experiments show that the promoter β-actin P2 has the ability to drive the expression of foreign gene in shrimp.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An isolated β-actin nucleic acid promoter molecule from shrimp having a nucleotide sequence comprising one or more (GC)ₙ-rich regions.

2. The isolated nucleic acid promoter molecule according to claim 1, wherein the nucleic acid promoter molecule has a nucleotide sequence of SEQ ID NO: 1.

3. A nucleic acid construct comprising:
a nucleic acid molecule encoding a protein;
the nucleic acid promoter molecule according to claim 1, wherein the nucleic acid promoter molecule is operably linked 5′ to the nucleic acid molecule encoding a protein to induce transcription of the nucleic acid molecule encoding a protein; and
a 3′ regulatory region operably linked to the nucleic acid molecule encoding a protein.

4. The nucleic acid construct according to claim 3, wherein the nucleic acid molecule encoding a protein has a sense orientation.

5. The nucleic acid construct according to claim 3, wherein
the nucleic acid molecule encoding a protein has an antisense orientation.

6. An expression vector comprising:
the nucleic acid construct according to claim 3.

7. A host cell transduced with the nucleic acid construct according to claim 3.

8. The host cell according to claim 7, wherein the cell is selected from the group consisting of a bacterial cell, a virus, a yeast cell, an insect cell, and a crustacean cell.

9. The host cell according to claim 8, wherein the cell is a shrimp cell.

10. A transgenic animal transformed with the nucleic acid construct according to claim 3.

11. The transgenic animal according to claim 10, wherein the animal is selected from the group consisting of marine fish, crustaceans, shellfish, and insects.

12. The transgenic animal according to claim 11, wherein the animal is a shrimp.

13. A nucleic acid expression cassette comprising:
the β-actin promoter molecule according to claim 1;
a multiple cloning site positioned in the nucleic acid construct to permit insertion of a nucleic acid molecule encoding a protein, whereby the nucleic acid molecule is transcribed;
an operable termination segment; and
a nucleic acid molecule encoding a detectable marker.

14. The nucleic acid expression cassette according to claim 13, wherein the detectable marker is selected from the group consisting of green fluorescent protein, enhanced green fluorescent protein, β-galactosidase, and luciferase.

15. A method of imparting to an animal resistance against a pathogen comprising:
transforming an animal with the nucleic acid construct according to claim 3, wherein the nucleic acid molecule encoding a protein encodes for resistance to a pathogen.

16. The method according to claim 15, wherein the pathogen is a virus.

17. The method according to claim 16, wherein the virus is selected from the group consisting of white spot syndrome virus, yellow head virus, Taura syndrome virus, and infectious hypodermal and hematopoietic necrosis virus.

18. The method according to claim 17, wherein the virus is Taura syndrome virus.

19. The method according to claim 17, wherein the virus is infectious hypodermal and hematopoietic necrosis virus.

20. The method according to claim 15, wherein the nucleic acid molecule encodes a viral coat protein or polypeptide.

21. The method according to claim 20, wherein the viral coat protein or polypeptide is selected from the group consisting of the coat protein or polypeptide of white spot syndrome virus, the coat protein or polypeptide of yellow head virus, the coat protein or polypeptide of Taura syndrome virus, and the coat protein or polypeptide of infectious hypodermal and hematopoietic necrosis virus.

22. The method according to claim 21, wherein the viral coat protein or polypeptide is the coat protein or polypeptide of Taura syndrome virus.

23. The method according to claim 21, wherein the viral coat protein or polypeptide is the coat protein or polypeptide of infectious hypodermal and hematopoietic necrosis virus.

24. The method according to claim 15, wherein the animal is selected from the group consisting of marine fish, crustaceans, shellfish, and insects.

25. The method according to claim 24, wherein the animal is a shrimp.

26. A transgenic animal prepared according to the method of claim 15, wherein the animal is selected from the group consisting of marine fish, crustaceans, shellfish, and insects.

27. The transgenic animal according to claim 26, wherein the animal is shrimp.

28. A method of regulating growth of an animal comprising:
transforming an animal with the nucleic acid construct according to claim 3, wherein the nucleic acid molecule encoding a protein encodes a growth regulating protein.

29. The method according to claim 28, wherein the growth regulating protein is a growth hormone.

30. The method according to claim 29, wherein the growth hormone is the androgenic hormone.

31. The method according to claim 28, wherein the animal is selected from the group consisting of marine fish, crustaceans, shellfish, and insects.

32. The method according to claim 31, wherein the animal is a shrimp.

33. A transgenic animal prepared according to claim 28, wherein the animal is selected from the group consisting of marine fish, crustaceans, shellfish, and insects.

34. The transgenic animal prepared according to the method of claim 33, wherein the animal is shrimp.

35. A method of increasing stress tolerance in an animal comprising:
transforming an animal with the nucleic acid construct according to claim 3, wherein the nucleic acid molecule encoding a protein encodes a nucleic acid capable of increasing stress tolerance.

36. The method according to claim 35, wherein the nucleic acid molecule encoding a protein encodes a heat shock protein.

37. A transgenic animal produced by the method according to claim 35.

38. The transgenic animal prepared according to claim 37, wherein the animal is selected from the group consisting of marine fish, crustaceans, shellfish, and insects.

39. The transgenic animal prepared according to the method of claim 38, wherein the animal is a shrimp.

40. The method according to claim 35, wherein the stress is cold.

41. The method according to claim 40, wherein the nucleic acid molecule encoding a protein encodes a heat shock protein.

42. The method according to claim 41, wherein the heat shock protein is HSP70 or HSP26.

43. A transgenic animal produced by the method according to claim 41.

44. The transgenic animal according to claim 43, wherein the animal is selected from the group consisting of marine fish, crustaceans, shellfish, and insects.

45. A transgenic animal according to claim 44, wherein the animal is a shrimp.

46. An isolated nucleic acid molecule encoding β-actin from shrimp, wherein the nucleic acid molecule either 1) has a nucleotide sequence of SEQ ID NO: 2; or 2) encodes a protein having SEQ ID NO: 3.

47. A nucleic acid construct comprising:
the isolated nucleic acid molecule according to claim 46; and
5′ and 3′ regulatory regions, wherein the regulatory regions are operably linked to the isolated nucleic acid molecule to allow expression of the nucleic acid molecule.

48. An expression vector comprising:
the nucleic acid construct according to claim 47.

49. A host cell transduced with the nucleic acid construct according to claim 47.

50. An isolated shrimp β-actin protein or polypeptide having an amino acid sequence of SEQ ID NO: 3.

51. An isolated actin nucleic acid promoter molecule from shrimp having a nucleotide sequence comprising (CATA)-rich repeats and (CACA)-rich repeats.

52. The isolated nucleic acid promoter molecule according to claim 51, wherein the nucleic acid promoter molecule has a nucleotide sequence of SEQ ID NO: 4.

53. A nucleic acid construct comprising:
a nucleic acid molecule encoding a protein;
the isolated nucleic acid promoter molecule according to claim 51, wherein the nucleic acid promoter molecule is operably linked 5′ to the nucleic acid molecule encoding a protein to induce transcription of the nucleic acid molecule; and
an operably linked 3′ regulatory region.

54. The nucleic acid construct according to claim 53, wherein the nucleic acid molecule encoding a protein has a sense orientation.

55. The nucleic acid construct according to claim 53, wherein the nucleic acid molecule encoding a protein has an antisense orientation.

56. An expression vector comprising:
the nucleic acid construct according to claim 53.

57. A host cell transduced with the nucleic acid construct according to claim 53.

58. The host cell according to claim 57, wherein the cell is selected from the group consisting of a bacterial cell, a virus, a yeast cell, an insect cell, and a crustacean cell.

59. The host cell according to claim 58, wherein the cell is a shrimp cell.

60. A transgenic animal transformed with the nucleic acid construct according to claim 53.

61. The transgenic animal according to claim 60, wherein the animal is selected from the group consisting of marine fish, crustaceans, shellfish, and insects.

62. The transgenic animal according to claim 61, wherein the animal is a shrimp.

63. A nucleic acid expression cassette comprising:
the actin promoter molecule according to claim 51;
a multiple cloning site positioned in the nucleic acid construct to permit insertion of a nucleic acid molecule encoding a protein, whereby the nucleic acid molecule is transcribed,
an operable termination segment; and
a nucleic acid molecule encoding a detectable marker.

64. The nucleic acid expression cassette according to claim 63, wherein the detectable marker is selected from the group consisting of green fluorescent protein, enhanced green fluorescent protein, β-galactosidase, and luciferase.

65. A method of imparting to an animal resistance against a pathogen comprising:
transforming an animal with the nucleic acid construct according to claim 53, wherein the nucleic acid molecule encoding a protein encodes for resistance to a pathogen.

66. The method according to claim 65, wherein the pathogen is a virus.

67. The method according to claim 66, wherein the virus is selected from the group consisting of white spot syndrome virus, yellow head virus, Taura syndrome virus, and infectious hypodermal and hematopoietic necrosis virus.

68. The method according to claim 67, wherein the virus is Taura syndrome virus.

69. The method according to claim 67, wherein the virus is infectious hypodermal and hematopoietic necrosis virus.

70. The method according to claim 66, wherein the nucleic acid molecule encodes a viral coat protein or polypeptide.

71. The method according to claim 70, wherein the viral coat protein or polypeptide is selected from the group consisting of the coat protein or polypeptide of white spot syndrome virus, the coat protein or polypeptide of yellow head virus, the coat protein or polypeptide of Taura syndrome virus, and the coat protein or polypeptide of infectious hypodermal and hematopoietic necrosis virus.

72. The method according to claim 71, wherein the viral coat protein or polypeptide is the coat protein or polypeptide of Taura syndrome virus.

73. The method according to claim 71, wherein the viral coat protein or polypeptide is the coat protein or polypeptide of infectious hypodermal and hematopoietic necrosis virus.

74. The method according to claim 65, wherein the animal is selected from the group consisting of marine fish, crustaceans, shellfish, and insects.

75. The method according to claim 74, wherein the animal is a shrimp.

76. A transgenic animal prepared according to the method of claim 65, wherein the animal is selected from the group consisting of marine fish, crustaceans, shellfish, and insects.

77. The transgenic animal according to claim 76, wherein the animal is a shrimp.

78. A method of regulating growth of an animal comprising:
transforming an animal with a nucleic acid construct according to claim 53, wherein the nucleic acid molecule encoding a protein encodes for a growth regulating protein.

79. The method according to claim 78, wherein growth regulating protein is a growth hormone.

80. The method according to claim 79, wherein the growth hormone is the androgenic hormone.

81. The method according to claim 78, wherein the animal is selected from the group consisting of marine fish, crustaceans, shellfish, and insects.

82. The method according to claim 81, wherein the animal is a shrimp.

83. A transgenic animal prepared according to the method of claim 78, wherein the animal is selected from the group consisting of marine fish, crustaceans, shellfish, and insects.

84. The transgenic animal according to claim 83, wherein the animal is shrimp.

85. A method of increasing stress tolerance in an animal comprising:
transforming an animal with a nucleic acid construct according to claim 53, wherein the nucleic acid molecule encoding a protein encodes for a protein capable of increasing stress tolerance in the animal.

86. The method according to claim 85, wherein the nucleic acid encodes a heat shock protein.

87. A transgenic animal produced by the method according to claim 85.

88. The transgenic animal according to claim 87, wherein the animal is selected from the group consisting of marine fish, crustaceans, shellfish, and insects.

89. The transgenic animal according to claim 88, wherein the animal is a shrimp.

90. The method according to claim 85, wherein the stress in cold.

91. The method according to claim 90, wherein the nucleic acid molecule encoding a protein encodes a heat shock protein.

92. The method according to claim 91, wherein the heat shock protein is HSP70 or HSP26.

93. A transgenic animal produced by the method according to claim 90.

94. The transgenic animal prepared according to the method of claim 90, wherein the animal is selected from the group consisting of marine fish, crustaceans, shellfish, and insects.

95. The transgenic animal according to claim 94, wherein the animal is a shrimp.

96. An isolated nucleic acid molecule encoding actin from shrimp, wherein the nucleic acid molecule either 1) has a nucleotide sequence of SEQ ID NO: 5; or 2) encodes a protein having SEQ ID NO: 6.

97. A nucleic acid construct comprising:
the isolated nucleic acid molecule according to claim 96; and
5′ and 3′ regulatory regions, wherein the regulatory regions are operably linked to the isolated nucleic acid molecule to allow expression of the nucleic acid molecule.

98. An expression vector comprising:
the nucleic acid construct according to claim 97.

99. A host cell transduced with the nucleic acid construct according to claim 97.

100. An isolated shrimp actin protein or polypeptide having an amino acid sequence of SEQ ID NO: 6.
